SEQUENCE LISTING (44 sequences)

SEQ ID NO: 1 - DNA, 1185 bp, Escherichia coli
  source 1..1185: mol_type = genomic DNA
atgaaaaattgtgtcatcgtcagtgcggtacgtactgctatcggtagttttaacggttcactcgcttccaccagcaccatcgacctgggggcgacagtaattaaagccgccattgaacgtgcaaaaatcgattcactacacgttgatgaagtgattatgggtaacgtgctacaagccgggctggggcaaaatccggcgcgtcaggcactgttaaaaagcggactggccgaaacagtgtgcggattcacggtcaataaagtgtgtggttcgggtcttaaaagtgtggcgcttgccgcccaggcaattcaggcaggtcaggcgcagagcattgtggcggggggtatggaaaatatgagtttagcgccctacttactcgatgcaaaagcacgctctggttatcgtcttggagacggacaggtttatgacgtaatcctgcgcgatggtctgatgtgcgccacccatggttatcatatggggattaccgccgaaaacgtggctaaagagtacggaattacccgtgaaatgcaggatgaactggcgctacattcacagcgtaaagcggcagccgcaattgagtccggtgcttttacagccgaaatcgtcccggtaaatgttgtcactcggaagaaaaccttcgtcttcagtcaagacgaattcccgaaagcggattctacggctgaagcgttaggcgcattgcgcccggccttcgataaagcaggaacagtcaccgccgggaacgcgtctggcattaacgacggtgctgccgctctggtgattatggaagaatcttcggcgctggcagcaggccttacccccctggctcgcattaaaagttatgccagcggtggcgtgccccccgcattgatgggtatggggccagtacctgccacgcaaaaagcgttacaactggcggggctgcaactggcggatattgatctcattgaggctaatgaagcatttgctgcacagttccttgccgttgggaaaaccctgggctttgagcctgagaaagtgaatgtcaacggcggggccatcgcgctggggcatcctatcggtgccagtggtgctcgtattctggtcacactattacatgcaatgcaggcacgcgataaaacgctggggctggcaacactgtgcattggtggcggccagggaattgcgatggtgattgaacgattgaattaa

SEQ ID NO: 2 - DNA, 4850 bp, Clostridium butyricum
  source 1..4850: mol_type = genomic DNA
atggaattagaaaatgtgattcttgaaaaagaaggacatttagcaattgttacaattaatagacctaaagctttaaatgctttaaattctgcgactttaaaggacttggatacagtattagaagatttagaaaatgatactaacatctatgctgttatcttaactggagcaggtgaaaaatcttttgtagctggagcagatattgcagaaatgaaagatttaaatgaagctcaaggaaaagaatttggagaacttggtaataaagtatttttaagattagaaaatttaaacaagcctgttattgcagcaatacaaggatttgctcttggtggcggatgcgaaatatctatggcatgtgacataagaatagcatctgaaactgcattatttggacaacctgaagtaggtttaggaattacaccaggatttggtggaactcaaagattagcaagaatagttggactaggaaaagctaaagaaatgatttatacagctagaaatattaaagctgatgaagcttatagaataggattagttaacaaagttgtagctttagaagatcttatgaatgaagctaaaaaaatggcttcaaatattatagcaaatgctccagtagctgttaagttatgtaaggatgcaataaacagaggtatgcaagtaggaatcgatgaagctgttatgatagaagctgaagattttggtaaatgttttgctacagaagatcaaactgaaggaatgactgctttcttagaaagaagaaaagaaaagaatttccaaaataaatagtattttaatatggtaaaaatccattacagatttatatatatgttatcaaggttttaattaacaaggagggcaatttaatgaatttccaattaactagagaacaagaattagtacaacaaatggttagagaattcgcagtaaatgaagttaagccaattgctgctgaaatcgatgaaactgaaagatttccaatggaaaatgttaaaaaaatgggtgaattaggaatgatgggaatcccattttctaaagaagttggaggagctggcggagatgttctttcttacataatttcagtagaagaattatcaaaggtttgtggtacaacaggagttatactttcagctcatacttcattatgtgcatcagtaataaatgaaaatggtacaccagatcaaaaagctaaatatttaccagatctttgctcaggtaaaaaaataggagctttcggtttaactgaaccaggtgctggtacagatgcagcaggtcaacaaactactgcaagattagaaggtgaccattatgtattaaatggttcaaaaatcttcataactaatggtggagttgctgaaacatttatagtttttgctatgactgataaatcaatgggaactagaggaatatcagcatttatagttgaaaagtcattcccaggattctcaattggtaagttagaaaacaaaatgggtattagagcatcttctactactgaattaatttttgaagattgtatagtaccaaaagaaaacttaattggtaaagaaggtaaaggcttcggaatagcaatgaaaactcttgatggaggaagaattggtattgcagctcaagctttaggtattgcagaaggtgcttttgatgaagcagttgcatacatgaaagaaagaaaacaattcaataaaccactttcagctttccaaggattacaatggtacattgctgaaatggacgttaagatagaagctgcaaaacacttagtatataaggctgcttggaagaaacaaaatggtataccttattcagttgatgcagcaagagctaaattatttgctgctgatgttgcaatggaagttacaactaaggctgtacaattatttggtggttatggatacactaaagaatatccagtagaaagaatgatgagagatgctaagataactgaaatctatgaaggaacttcagaagttcaaaagatggttatcgctggaagcattttaagataggaggacttttagaatgaatatagtagtttgtgtaaaacaagttccagatacaacagctgtaaaaattgatccaaagactggtacattaataagagatggggttccatcaataataaatccagaggataaacatgctttagaaggtgcgttaagaataaaagaagcaactggtgctaaagtaacagttgtaagcatggggcttccaatggctaaagcagctcttagagaagcattatgtatgggagctgatgaagctattttattaacagatagagcacttggaggagctgatacattagcaacttcaaaagcattatcaggagttatagctaaattagatgctgatatagtattcgctggtagacaagcaattgatggagatactgcacaagttggtccagaaatcgctgaacatttagatattcctcaagtaacttacgttcaagacgtaaaagttgaaggggaaacattgatagtaaacagagcattagaagatggtcatcaagtagttgaagttaagactccatgtctattaactgcaattgaagaattaaatagcccaagatatatgaacgttgcaagaatatttgaaactaacgatgatgaaatcaaagttatgggagcagcagatatagatgtacctgttgaagaattaggacttaaaggttcgcctacaaaggttaagaagtcaatgactaaagaagttaaaggtcaaggtgaattagttaaacaagaacctaaagacgctgtaacatacgttttaggaaaattaaaagagaaacactacatctaagataataggaggttaagtaattatgaatatagcagattacaagggcgtttgggtctttgctgaacaaagagaaggcgaattacaaaaagtgtctttagaattacttggagaaggaagaagacaagctgataaactaggagttaagttaacagcattattattaggaagcaatattgaaggattaagcaaaacattagcagaacatggtgctgatgaagttttaattgctgatgataaattattagaacattatacaactgatgcatatacaaaagttatctgtgacctagtaaatgaaagaaaaccaggaatattatttgttggtgctacttttatcggaagagatttaggtccaagagttgcagctagattaaacacaggacttacagctgactgtacatcaatagatgtagaagttgaaaatggtgatttattagctacaagaccagcatttggtggtaacttaatggctacaattgcatgtcctgaacatagaccacaaatggctacagtaagaccaggagtttttgctaaagttgaaagtgatggagctaactgtaacatagaaaaagttaatgtggaattaactgattctgatgttagaactaaagtattagaaacagttaaaactgctaaagatatcatagacatatcagaagctaaagttattgtttctggtggtagaggtgttggatctaaggaaaactttgcattactagaagaattagctaacatattaggtggaacagttgctggttctagagcagcagttgaaaaaggatggatagaaaatgcttaccaagttggacaaactggtaaaactgtaaaaccatcaatttatatagcatgtggtatttcaggagctatccaacacgttgctggtatgcaagattctgatatgattattgctgtaaataaagatgaaactgcgccaataatgaaggttgcagattacgctatcgttggagatattaaaaaggtattacctgaattaattgctcaagcaaaggaaattgtagaagctgaataattaattgattatagaaaaacagtagtggattttagagcggaatttaattaataaatttatataaaattttggttaatatattatgtatttactatatctaaggccagaaatatgcctggtcttagtattttaaataaatggaacagaaatatatatatttaggaggaatttattcatgaaaaaagtatttgtacttggtgcaggaactatgggtgctggaattgttcaagcatttgctgcaaaaggttgtgaagtaattgtaagagacataaaagaagaatttgttgatagaggaatagctacaataactaagtctttatctaaattagtggctaaagaaaaaattactgaagcagacaaagaagaaattctttcaagaatatcaggaacaactgatatgaaattagctgctgactgcgatttagtagttgaagctgctattgaaaacatgaaaattaaaagagaaatatttgctgaattagatgaaatctgtaaaccagaaactattttagcttcaaatacttcttcattatcaataactgaagttgcatctgcaactaagagagctgataaagttataggaatgcatttctttaatccagctccagttatgaagttagttgaagttataagaggagctgctacttctcaagaaacttttgatgctgttaaggaaatgtcagaatcaataggtaaaactccagtagaagttgctgaagcaccaggatttgttgtaaacagaattttaatccctatgattaatgaagctactttcattttacaagaaggtgttgcaaaggaagaagatatcgatgcagctatgaaacttggagctaaccacccaatgggacctttagctttaggtgaccttataggattagacgtatgtttagctatcatggacgttttatataacgaaacaggagatagtaaatacagagctagcagtcttttaagaaagtatgttagagctggttggttaggtagaaagactggaaaaggattctacgattattctaaataa

SEQ ID NO: 3 - DNA, 2072 bp, Clostridium acetobutylicum
  source 1..2072: mol_type = genomic DNA
atgagtaaaaattttgatgatttattttcaagattacaagaagttgaaactaaaaaagtagctgttgcagtagctcaagacgaacctgttttagaagctgtaaaagaagctaatgaaaagggaatagctaatgctgtattagtaggagataaagataagattcatgaaattgcaaaaaaaatagatatggatttaactaaatttgaaataatggatgttaaagatccaaagaaagcaactatggaggcagttaagcttgtatcaagcgggaatgctgacatgttaatgaaaggtttaatagatacagctacatttttaagaagtgtattaaataaagaggttggtcttagaactggtaaagtaatgtcccatgtatctgtatttgaaatagaaggatgggatagattattcttcttaacagacgttgcattcaatacttatccagaattaaaagataaagttacaattattaataatgctgtttcagttgcacatgcttgtggccttgatatgccaaaggttggggttgtatgtccagttgaagttgtaaatcctaatatgccatcgactgttgatgcagcattacttgcaaaaatgagcgatagaggacaatttaagggatgcgttgttgatggaccatttgctttagataatgcaatatctttagaagcagctgaacataaaggcgttaaaggtgaagttgcaggacaagctgatattttagtgatgccaaatattgaaacaggaaatgtaatgtataaaacattaacttattttgcaccagcaaaaaatggatgtcttttagttggaacatcagcaccagcaattcttacttcaagagcagatacttttgaaacaaaagttaattcaattgcattagcagcattagttgcagctaaaaataaatagtagaatattaaatgtaaactttaggatataattcttgggaatttatattaaatcacagtgtagatagattttaatgaaaatatggaggaaagaaaatggcttacaaattattaataattaatccaggttcaacatcaacaaaaataggtgtatatgaagatgaaaaagaattatttgaagaaacattaagacatacaaatgaagaattaaagcagtttgatgcgatttttgatcaatttcaatttagaaaagatgtaattttaaaagttttaagtgaaaaaaactttgatataaaaacattaagtgctgttgttggtagaggtggaatgcttaaaccagttgaaggtggtacttatgctgtaaatgatgcaatggtagaagatttaaaggtaggagttcaaggacctcatgcttctaacttaggaggtatacttgcaagatcaatagctgatgaaataggagtaccatctttcattgttgatccagttgttacagatgaacttgcagatgttgcaagattatctggtacaccagatattccaagaaaaagtaagttccatgcattaaatcaaaaggcagttgcaaagagatatggaaaagaaagtggaaaaggatatgaaaatttaaatcttgttgttgtacacatgggtggtggagtttctgttggagctcacaatcatggaaaagttgttgatgtaaacaatgcacttgatggagatggtccattttctccagaaagagctggatcagttccagctggagatttaattaaaatgtgtttcagtggtaagtatagcgaatcagaagtatacagcaaaatagttggtaaaggtggattcgttggatatttaaatactaatgatgtaaaaggaactattgataagatggaagctggagataaggaatgtgaaaacatatataaagctttcttatatcaaataacaaaagcaattggagaaatgtcagctgcgttaaatggtaaagttgatcagatagttttaacaggtggtattgcatattctccaacacttgtaccagatttaaaagctaatgttgaatggattgcaccagtaacagtatatccaggagaagatgaattgttagctttagctcaaggtgctataagagttcttgatggagaagaaaaggctaaagtttattaa

SEQ ID NO: 4 - DNA, 990 bp, Escherichia coli
  source 1..990: mol_type = genomic DNA
atgaaactcgccgtttatagcacaaaacagtacgacaagaagtacctgcaacaggtgaacgagtcctttggctttgagctggaattttttgactttctgctgacggaaaaaaccgctaaaactgccaatggctgcgaagcggtatgtattttcgtaaacgatgacggcagccgcccggtgctggaagagctgaaaaagcacggcgttaaatatatcgccttgcgctgtgccggtttcaataacgtcgaccttgacgcggcaaaagaactggggctcaaagtagtccgtgttccagcctatgatccagaggccgttgctgaacacgccatcggtatgatgatgacgctgaaccgccgtattcaccgcgcatatcagcgtacccgtgacgctaacttctctctggaaggtctgaccggctttactatgtatggcaaaacggcaggcgttatcggtaccggtaaaatcggtgtggcaatgctgcgcattctgaaaggttttggtatgcgtctgctggcgttcgatccgtatccaagtgcggcggcgctggaactcggtgtggagtatgtcgatctgccaaccctgttctctgaatcagacgttatctctctgcactgcccgctgacaccggaaaactaccatctgttgaacgaagccgccttcgatcagatgaaaaatggcgtgatgatcgtcaataccagtcgcggtgcattgattgattctcaggcggcaattgaagcgctgaaaaatcagaaaattggttcgttgggtatggacgtgtatgagaacgaacgcgatctgttctttgaagataaatccaacgacgtgatccaggatgacgtattccgtcgcttgtctgcctgccacaacgtgttgtttaccgggcaccaggcattcctgacagcagaagctctgaccagtatttctcagactacgctgcaaaacttaagcaatctggaaaaaggcgaaacctgcccgaacgaactggtttaa

SEQ ID NO: 5 - DNA, 3312 bp, Escherichia coli
  source 1..3312: mol_type = genomic DNA
gtgcaaacctttcaagccgatcttgccattgtaggcgccggtggcgcgggattacgtgctgcaattgctgccgcgcaggcaaatccaaatgcaaaaatcgcactaatctcaaaagtatacccgatgcgtagccataccgttgctgcagaagggggctccgccgctgtcgcgcaggatcatgacagcttcgaatatcactttcacgatacagtagcgggtggcgactggttgtgtgagcaggatgtcgtggattatttcgtccaccactgcccaaccgaaatgacccaactggaactgtgggggtgcccatggagccgtcgcccggatggtagcgtcaacgtacgtcgcttcggcggcatgaaaatcgagcgcacctggttcgccgccgataagaccggcttccatatgctgcacacgctgttccagacctctctgcaattcccgcagatccagcgttttgacgaacatttcgtgctggatattctggttgatgatggtcatgttcgcggcctggtagcaatgaacatgatggaaggcacgctggtgcagatccgtgctaacgcggtcgttatggctaccggcggtgcgggtcgcgtttatcgttacaacaccaacggcggcatcgttaccggtgacggtatgggtatggcgctaagccacggcgttccgctgcgtgacatggaattcgttcagtatcacccaaccggtctaccaggttccggtatcctgatgaccgaaggctgccgcggtgaaggtggtattctggtcaacaaaaatggctaccgttatctgcaagattacggcatgggcccggaaactccgctgggcgagccgaaaaacaaatatatggaactgggtccacgcgacaaagtttctcaggccttctggcacgaatggcgtaaaggcaacaccatctccacgccacgtggcgatgtggtttacctcgacctgcgtcacctcggcgagaaaaaactgcatgaacgtctgccgttcatctgcgaactggcgaaagcgtacgttggcgtcgatccggttaaagaaccgattccggtacgtccgaccgcacactacaccatgggcggtatcgaaaccgatcagaactgtgaaacccgcattaaaggtctgttcgccgtgggtgaatgttcctctgttggtctgcacggtgcgaaccgtctgggctccaactcgctggcggaactggtggtcttcggtcgtctggccggtgaacaagcgacagagcgtgcagcaactgccggtaatggcaacgaagcggcaattgaagcgcaggcagctggcgttgaacaacgtctgaaagatctggttaaccaggatggcggcgaaaactgggctaagatccgcgacgaaatgggcatggcaatggaagaaggttgcggtatctaccgtacgccggaactgatgcagaaaaccatcgacaagctggcagagctgcaggaacgcttcaagcgcgtgcgcatcaccgacacttccagcgtgttcaacaccgacctgctctacaccattgagctgggccacggtctgaacgttgctgaatgtatggcgcactccgcaatggcacgtaaagagtcccgcggcgcgcaccagcgtctggacgaaggttgcaccgagcgtgacgacgtcaacttcctcaaacacaccctcgccttccgcgatgctgatggcacgactcgcctggagtacagcgacgtgaagattactacgctgccgccagctaaacgtgtttacggtggcgaagcggatgcagccgataaggcggaagcagccaataagaaggagaaggcgaatggctgagatgaaaaacctgaaaattgaggtggtgcgctataacccggaagtcgataccgcaccgcatagcgcattctatgaagtgccttatgacgcaactacctcattactggatgcgctgggctacatcaaagacaacctggcaccggacctgagctaccgctggtcctgccgtatggcgatttgtggctcctgcggcatgatggttaacaacgtgccaaaactggcatgtaaaaccttcctgcgtgattacaccgacggtatgaaggttgaagcgttagctaacttcccgattgaacgcgatctggtggtcgatatgactcacttcatcgaaagtctggaagcgatcaaaccgtacatcatcggcaactcccgcaccgcggatcagggtactaacatccagaccccggcgcagatggcgaagtatcaccagttctccggttgcatcaactgtggtctgtgctacgccgcgtgcccgcagtttggcctgaacccagagttcatcggtccggctgccattacgctggcgcatcgttataacgaagatagccgcgaccacggtaagaaggagcgtatggcgcagttgaacagccagaacggcgtatggagctgtactttcgtgggctactgctccgaagtctgcccgaaacacgtcgatccggctgcggccattcagcagggcaaagtagaaagttcgaaagactttcttatcgcgaccctgaaaccacgctaaggagtgcaacatgacgactaaacgtaaaccgtatgtacggccaatgacgtccacctggtggaaaaaattgccgttttatcgcttttacatgctgcgcgaaggcacggcggttccggctgtgtggttcagcattgaactgattttcgggctgtttgccctgaaaaatggcccggaagcctgggcgggattcgtcgactttttacaaaacccggttatcgtgatcattaacctgatcactctggcggcagccctgctgcacaccaaaacctggtttgagctggcaccaaaagcagccaatatcattgtaaaagacgaaaaaatgggaccagagccaattatcaaaagtctctgggcggtaactgtggttgccaccatcgtaatcctgtttgttgccctgtactggtaaggagcctgagatgattaatccaaatccaaagcgttctgacgaaccggtattctggggcctcttcggggccggtggtatgtggagcgccatcattgcgccggtgatgatcctgctggttggtattctgctgccgctagggctgtttccgggtgatgcactgagctacgagcgcgttctggcgttcgcgcagagcttcattggtcgcgtattcctgttcctgatgatcgttctgccgctgtggtgtggtttacaccgtatgcaccacgcgatgcacgatctgaaaatccacgtacctgcgggtaaatgggttttctacggtctggctgctatcctgacagttgtcacgctgattggtgtcgttacaatctaa

SEQ ID NO: 6 - DNA, 2676 bp, Escherichia coli
  source 1..2676: mol_type = genomic DNA
atggctgttactaatgtcgctgaacttaacgcactcgtagagcgtgtaaaaaaagcccagcgtgaatatgccagtttcactcaagagcaagtagacaaaatcttccgcgccgccgctctggctgctgcagatgctcgaatcccactcgcgaaaatggccgttgccgaatccggcatgggtatcgtcgaagataaagtgatcaaaaaccactttgcttctgaatatatctacaacgcttataaagatgaaaaaacctgtggtgttctgtctgaagacgacacttttggtaccatcactatcgctgaacccatcggtattatttgcggtatcgttccgaccactaacccgacttcaactgctatcttcaaatcgctgatcagcctgaagacccgtaacgccattatcttctccccgcacccgcgtgcaaaagatgcaaccaacaaagcggctgatatcgttctacaggctgctatcgctgccggtgctccgaaagatctgatcggctggatcgatcaaccttctgttgagctgtctaacgcactgatgcaccacccagacatcaacctgatcctcgcgactggtggtccgggcatggttaaagccgcatacagctccggtaaaccagctatcggcgtaggcgcgggcaacactccggttgttatcgatgaaactgctgatatcaaacgtgcagttgcatctgtactgatgtccaaaaccttcgacaacggtgtaatctgtgcttctgaacagtctgttgttgttgttgactctgtttatgacgcagtacgtgaacgtttcgcaacccacggcggctatctgttgcagggtaaagagctgaaagctgttcaggacgttatcctgaaaaacggtgcgctgaacgcggctatcgttggtcagccagcctataaaattgctgaactggcaggcttctctgtaccagaaaacaccaagattctgatcggtgaagtgaccgttgttgatgaaagcgaaccgttcgcacatgaaaaactgtccccgactctggcaatgtaccgtgctaaagatttcgaagacgcggtagaaaaagcagagaaactggttgctatgggcggtatcggtcatacctcttgcctgtacactgaccaggataaccaaccggctcgcgtttcttacttcggtcagaaaatgaaaacggctcgtatcctgattaacaccccggcttctcagggtggtatcggtgacctgtataacttcaaactcgcaccttccctgactctgggttgtggttcctggggtggtaactccatctctgaaaacgttggtccgaaacacctgatcaacaagaaaaccgttgctaagcgagctgaaaacatgttgtggcacaaacttccgaaatctatctacttccgccgtggctccctgccaatcgcgctggatgaagtgattactgatggccacaaacgtgcgctcatcgtgactgaccgcttcctgttcaacaatggttatgctgatcagatcacttccgtattgaaagcagcaggcgttgaaactgaagtcttcttcgaagtagaagctgacccgaccctgagcatcgttcgtaaaggtgcagaactggcaaactccttcaaaccagacgtgattatcgcgctgggtggaggttccccgatggacgctgcgaagatcatgtgggttatgtacgaacatccggaaactcacttcgaagaactggcgctgcgctttatggatatccgtaaacgtatctacaagttcccgaaaatgggtgtgaaagcgaaaatgatcgctgtcaccaccacttctggtacaggttctgaagtcactccgtttgcggttgtaactgacgacactactggtcagaaatatccgctggcagactatgcactgaccccggatatggcgattgtcgacgccaacctggttatggacatgccgaagtccctgtgtgctttcggtggtctggacgcagtaactcacgccatggaagcttatgtttctgtactggcatctgagttctctgatggtcaggctctgcaggcactgaaactgctgaaagaatatctgccagcgtcctaccacgaagggtctaaaaatccggtagcgcgtgaacgtgttcacagtgcagcgactatcgcgggtatcgcgtttgcgaacgccttcctgggtgtatgtcactcaatggcgcacaaactgggttcccagttccatattccgcacggtctggcaaacgccctgctgatttgtaacgttattcgctacaacgcgaatgacaacccgaccaagcagactgcattcagccagtatgaccgtccgcaggctcgccgtcgttatgctgaaattgctgaccacctgggtctgagcgcaccgggcgaccgtactgcagctaagatcgagaaactgctggcatggctggaaacgctgaaagctgaactgggtattccgaaatctatccgtgaagctggcgttcaggaagcagacttcctggcgaacgtggataaactgtctgaagatgcattcgatgaccagtgcaccggcgctaacccgcgttacccgctgatctccgagctgaaacagattctgctggatacctactacggtcgtgattatgtagaaggcgaaactgcagcgaagaaagaagctgctccggctaaagctgagaaaaaagcgaaaaaatccgcttaa

SEQ ID NO: 7 - DNA, 1203 bp, Escherichia coli
  source 1..1203: mol_type = genomic DNA
atgtcgagtaagttagtactggttctgaactgcggtagttcttcactgaaatttgccatcatcgatgcagtaaatggtgaagagtacctttctggtttagccgaatgtttccacctgcccgaagcacgtatcaaatggaaaatggacggcaataaacaggaagcggctttaggtgcaggcgccgctcacagcgaagcgctcaactttatcgttaatactattctggcacaaaaaccagaactgtctgcgcagctgactgctatcggtcaccgtatcgtacacggcggcgaaaagtataccagctccgtagtgatcgatgagtctgttattcagggtatcaaagatgcagcttcttttgcaccgctgcacaacccggctcacctgatcggtatcgaagaagctctgaaatctttcccacagctgaaagacaaaaacgttgctgtatttgacaccgcgttccaccagactatgccggaagagtcttacctctacgccctgccgtacaacctgtacaaagagcacggcatccgtcgttacggcgcgcacggcaccagccacttctatgtaacccaggaagcggcaaaaatgctgaacaaaccggtagaagaactgaacatcatcacctgccacctgggcaacggtggttccgtttctgctatccgcaacggtaaatgcgttgacacctctatgggcctgaccccgctggaaggtctggtcatgggtacccgttctggtgatatcgatccggcgatcatcttccacctgcacgacaccctgggcatgagcgttgacgcaatcaacaaactgctaaccaaagagtctggcctgctgggtctgaccgaagtgaccagcgactgccgctatgttgaagacaactacgcgacgaaagaagacgcgaagcgcgcaatggacgtttactgccaccgcctggccaaatacatcggtgcctacactgcgctgatggatggtcgtctggacgctgttgtattcaccggtggtatcggtgaaaatgccgcgatggttcgtgaactgtctctgggcaaactgggcgtgctgggctttgaagttgatcatgaacgcaacctggctgcacgtttcggcaaatctggtttcatcaacaaagaaggtacccgtcctgcggtggttatcccaaccaacgaagaactggttatcgcgcaagacgcgagccgcctgactgcctga

SEQ ID NO: 8 - DNA, 2145 bp, Escherichia coli
  source 1..2145: mol_type = genomic DNA
gtgtcccgtattattatgctgatccctaccggaaccagcgtcggtctgaccagcgtcagccttggcgtgatccgtgcaatggaacgcaaaggcgttcgtctgagcgttttcaaacctatcgctcagccgcgtaccggtggcgatgcgcccgatcagactacgactatcgtgcgtgcgaactcttccaccacgacggccgctgaaccgctgaaaatgagctacgttgaaggtctgctttccagcaatcagaaagatgtgctgatggaagagatcatcgcgaactaccacgctaacaccaaagacgctgaagtcgttctggtggaaggtctggtcccgacacgtaagcaccagtttgcccagtctctgaactacgaaatcgccaaaacgctgaacgcagaaatcgtcttcgttatgtctcagggcactgatactccggaacagttgaaagagcgtatcgaactgactcgcaacagcttcggcggtgcaaaaaacaccaatattaccggcgttatcgttaacaaactgaacgctccggttgatgagcagggtcgtacccgtccggatctgtccgagatttttgacgactccaccaaagcaaaagtgaacaacgttgatccggcgaagctgcaagaatccagcccgctgccggttctcggcgctgtgccgtggagctttgacctgatcgcgactcgtgcgatcgatatggctcgccacctgaatgcgaccatcatcaacgaaggcgacatcaatactcgccgcgttaaatccgtcactttctgcgcacgcagcattccgcacatgctggagcacttccgtgccggttctctgctggtgacttccgcagaccgccctgacgtgctggttgccgcttgcctggctgccatgaacggcgtagaaatcggtgccctgctgctgactggcggctacgaaatggacgcgcgcatttctaaactgtgcgaacgtgctttcgctactggcctgccggtatttatggtgaacaccaacacctggcagacttctcttagcctgcagagcttcaacctggaagttccggttgacgatcatgagcgtatcgaaaaagttcaggaatacgtggctaactacatcaacgctgactggatcgattctctgactgccacttctgagcgcagccgtcgtctgtctccgccagcgttccgttatcagctgactgaacttgcgcgcaaagcgggcaaacgtatcgttctgccggaaggtgacgaaccgcgtaccgttaaagcagccgctatctgtgctgaacgtggtatcgcaacttgcgtactgctgggtaatccggcagagatcaaccgtgttgcagcctctcagggtgtagaactgggtgcaggcattgaaatcgttgatccagaagtggttcgcgaaaactatgttggtcgtctggtcgaactgcgtaagaacaaaggcatgaccgaaaccgttgcccgcgaacagctggaagacaacgtggttctcggtacgctgatgctggaacaagatgaagttgatggtctggtttccggtgctgttcacaccaccgcaaacaccatccgtccgccgctgcagctgatcaaaactgcaccgggcagctccctggtatcttccgtgttcttcatgctgttgccggaacaggtttacgtttacggtgactgtgcgatcaacccggatccgaccgcagaacagctggcagaaatcgcgattcagtccgctgattccgctgcggccttcggtatcgaaccgcgcgttgctatgctctcctactccaccggtacttctggtgctggtagcgacgtagaaaaagttcgcgaagcaactcgtctggcgcaggaaaaacgtcctgatctgatgatcgacggtccgctgcagtacgacgctgcggtaatggctgacgttgcgaaatccaaagcaccgaactctccggttgcaggtcgcgctaccgtgttcatcttcccggatctgaacaccggtaacaccacctacaaagcggtacagcgttctgctgacctgatctctatcggaccgatgctgcagggtatgcgcaagccggttaacgacctgtcccgtggcgcactggttgatgatatcgtctacaccatcgcgctgactgcgattcagtctgcacagcagcagtaa

SEQ ID NO: 9 - DNA, 30 bp, synthetic construct
  source 1..30: mol_type = other DNA
  misc_feature 1..30: note = PCR_primers_fwd_seq
atcggctagctgatgcggcaacaatcacac

SEQ ID NO: 10 - DNA, 30 bp, synthetic construct
  source 1..30: mol_type = other DNA
  misc_feature 1..30: note = PCR_primers_rev_seq
tgcagctagcagtcagaccagcatcagtcc

SEQ ID NO: 11 - DNA, 47 bp, synthetic construct
  source 1..47: mol_type = other DNA
  misc_feature 1..47: note = PCR_primers_fwd_seq
tagagtttaaggagatatacatatgtctaacgtgcaggagtggcaac

SEQ ID NO: 12 - DNA, 48 bp, synthetic construct
  source 1..48: mol_type = other DNA
  misc_feature 1..48: note = PCR_primers_rev_seq
cagcggtggcagcagcctaggttaattaacccagcatcgagccggttg

SEQ ID NO: 13 - DNA, 55 bp, synthetic construct
  source 1..55: mol_type = other DNA
  misc_feature 1..55: note = PCR_primers_fwd_seq
aatatcccgttaaataaatataggaggttaagtaatgaaaaattgtgtcatcgtc

SEQ ID NO: 14 - DNA, 42 bp, synthetic construct
  source 1..42: mol_type = other DNA
  misc_feature 1..42: note = PCR_primers_rev_seq
gctagttattgctcagcggtttaattcaatcgttcaatcacc

SEQ ID NO: 15 - DNA, 25 bp, synthetic construct
  source 1..25: mol_type = other DNA
  misc_feature 1..25: note = PCR_primers_fwd_seq
gcttctaggagtatatttatttaac

SEQ ID NO: 16 - DNA, 16 bp, synthetic construct
  source 1..16: mol_type = other DNA
  misc_feature 1..16: note = PCR_primers_rev_seq
accgctgagcaataac

SEQ ID NO: 17 - DNA, 46 bp, synthetic construct
  source 1..46: mol_type = other DNA
  misc_feature 1..46: note = PCR_primers_fwd_seq
attaatcagataaaatatttattttcagaaaatttagcatttaaag

SEQ ID NO: 18 - DNA, 45 bp, synthetic construct
  source 1..45: mol_type = other DNA
  misc_feature 1..45: note = PCR_primers_rev_seq
actgagctagctgtaaagaacatttttataaattccattttttcc

SEQ ID NO: 19 - DNA, 75 bp, synthetic construct
  source 1..75: mol_type = other DNA
  misc_feature 1..75: note = PCR_primers_fwd_seq
atgaaactcgccgtttatagcacaaaacagtacgacaagaagtacctgcaaatatcctccttagttcctattccg

SEQ ID NO: 20 - DNA, 74 bp, synthetic construct
  source 1..74: mol_type = other DNA
  misc_feature 1..74: note = PCR_primers_rev_seq
ttaaaccagttcgttcgggcaggtttcgcctttttccagattgcttaagtctgcttcgaagttcctatactttc

SEQ ID NO: 21 - DNA, 75 bp, synthetic construct
  source 1..75: mol_type = other DNA
  misc_feature 1..75: note = PCR_primers_fwd_seq
atggctgttactaatgtcgctgaacttaacgcactcgtagagcgtgtaaaaatatcctccttagttcctattccg

SEQ ID NO: 22 - DNA, 74 bp, synthetic construct
  source 1..74: mol_type = other DNA
  misc_feature 1..74: note = PCR_primers_rev_seq
ttaagcggattttttcgcttttttctcagctttagccggagcagcttcttctgcttcgaagttcctatactttc

SEQ ID NO: 23 - DNA, 59 bp, synthetic construct
  source 1..59: mol_type = other DNA
  misc_feature 1..59: note = PCR_primers_fwd_seq
tgtcaaatatgaatttctccagatacgtaaatctatgagcgaacttcagagcgcttttg

SEQ ID NO: 24 - DNA, 60 bp, synthetic construct
  source 1..60: mol_type = other DNA
  misc_feature 1..60: note = PCR_primers_rev_seq
aatatgtattgcctgaatagtaaagtctttttgggggtgtggtcacagcttgtctgtaag

SEQ ID NO: 25 - DNA, 75 bp, synthetic construct
  source 1..75: mol_type = other DNA
  misc_feature 1..75: note = PCR_primers_fwd_seq
taaaaaaagcacgatctgatggtttagtaattaaattaatcatcttcagtaatatcctccttagttcctattccg

SEQ ID NO: 26 - DNA, 74 bp, synthetic construct
  source 1..74: mol_type = other DNA
  misc_feature 1..74: note = PCR_primers_rev_seq
gttgcgtcataaggcacttcatagaatgcgctatgcggtgcggtatcgacctgcttcgaagttcctatactttc

SEQ ID NO: 27 - DNA, 61 bp, synthetic construct
  source 1..61: mol_type = other DNA
  misc_feature 1..61: note = PCR_primers_fwd_seq
tgtaacccgccaaatcggcggtaacgaaagaggataaaccgtgtcccgtattattatgctg

SEQ ID NO: 28 - DNA, 57 bp, synthetic construct
  source 1..57: mol_type = other DNA
  misc_feature 1..57: note = PCR_primers_rev_seq
ttcagatatccgcagcgcaaagctgcggatgatgacgagattactgctgctgtgcag

SEQ ID NO: 29 - DNA, 21 bp, synthetic construct
  source 1..21: mol_type = other DNA
  misc_feature 1..21: note = PCR_primers_fwd_seq
agaagagcccgtcctctgtga

SEQ ID NO: 30 - DNA, 20 bp, synthetic construct
  source 1..20: mol_type = other DNA
  misc_feature 1..20: note = PCR_primers_rev_seq
tcagacagcacgaggcattt

SEQ ID NO: 31 - DNA, 20 bp, synthetic construct
  source 1..20: mol_type = other DNA
  misc_feature 1..20: note = PCR_primers_fwd_seq
gcatgatccgagatgtggaa

SEQ ID NO: 32 - DNA, 20 bp, synthetic construct
  source 1..20: mol_type = other DNA
  misc_feature 1..20: note = PCR_primers_rev_seq
cagacaccgcctggagttct

SEQ ID NO: 33 - DNA, 20 bp, synthetic construct
  source 1..20: mol_type = other DNA
  misc_feature 1..20: note = PCR_primers_fwd_seq
gaattccctgggagagaagc

SEQ ID NO: 34 - DNA, 20 bp, synthetic construct
  source 1..20: mol_type = other DNA
  misc_feature 1..20: note = PCR_primers_rev_seq
cgggtggttcaatttttcat

SEQ ID NO: 35 - DNA, 20 bp, synthetic construct
  source 1..20: mol_type = other DNA
  misc_feature 1..20: note = PCR_primers_fwd_seq
cacttcacaagtcggaggct

SEQ ID NO: 36 - DNA, 20 bp, synthetic construct
  source 1..20: mol_type = other DNA
  misc_feature 1..20: note = PCR_primers_rev_seq
tctgacagtgcatcatcgct

SEQ ID NO: 37 - DNA, 20 bp, synthetic construct
  source 1..20: mol_type = other DNA
  misc_feature 1..20: note = PCR_primers_fwd_seq
tccccagggacctctctcta

SEQ ID NO: 38 - DNA, 22 bp, synthetic construct
  source 1..22: mol_type = other DNA
  misc_feature 1..22: note = PCR_primers_rev_seq
gggtttgctacaacatgggcta

SEQ ID NO: 39 - DNA, 23 bp, synthetic construct
  source 1..23: mol_type = other DNA
  misc_feature 1..23: note = PCR_primers_fwd_seq
atgatggcttattacagtggcaa

SEQ ID NO: 40 - DNA, 20 bp, synthetic construct
  source 1..20: mol_type = other DNA
  misc_feature 1..20: note = PCR_primers_rev_seq
gtcggagattcgtagctgga

SEQ ID NO: 41 - DNA, 21 bp, synthetic construct
  source 1..21: mol_type = other DNA
  misc_feature 1..21: note = PCR_primers_fwd_seq
gaagatgtgcctgtcctgtgt

SEQ ID NO: 42 - DNA, 21 bp, synthetic construct
  source 1..21: mol_type = other DNA
  misc_feature 1..21: note = PCR_primers_rev_seq
cgctcaggtcagtgatgttaa

SEQ ID NO: 43 - DNA, 23 bp, synthetic construct
  source 1..23: mol_type = other DNA
  misc_feature 1..23: note = PCR_primers_fwd_seq
gagaataagctgtaccatcgcaa

SEQ ID NO: 44 - DNA, 21 bp, synthetic construct
  source 1..21: mol_type = other DNA
  misc_feature 1..21: note = PCR_primers_rev_seq
cgaccacataggaagtcccag

(19) United States
(12) Patent Application Publication
(10) Pub. No.: US 2025/0161372 A1
(43) Pub. Date: May 22, 2025

(54) ENGINEERED PROBIOTICS AND THE APPLICATIONS THEREOF

(71) Applicant: City University of Hong Kong, Hong Kong (HK)

(72) Inventors: Quynh Hoa PHAM, Hong Kong (HK); Chi Kong LAU, Hong Kong (HK); Kiwon BAN, Hong Kong (HK); Thi Van Anh BUI, Hong Kong (HK)

(21) Appl. No.: 18/919,445

(22) Filed: Oct. 18, 2024

Related U.S. Application Data
(60) Provisional application No. 63/602,348, filed on Nov. 22, 2023.

(51) Int. Cl. A61K 35/741; A61K 38/44; A61K 38/45; A61K 38/51; A61K 47/46; A61P 9/10; C12N 15/52

(52) U.S. Cl. CPC A61K 35/741; A61K 38/443; A61K 38/45; A61K 38/51; A61K 47/46; A61P 9/10; C12N 15/52; C12Y 101/01157; C12Y 103/99002; C12Y 203/01009; C12Y 203/01019; C12Y 207/02007; C12Y 402/01017

(57) ABSTRACT

A genetically engineered strain of Escherichia coli Nissle 1917 (EcN) with a modified genome designed to enhance the production of short-chain fatty acids (SCFAs) is provided. The engineered genome includes the atoB gene from E. coli K-12, responsible for encoding acetyl-CoA acetyltransferase, a crt-bcd-etfA-etfB-BHBD gene cluster from E. C. butyricum that encodes enzymes involved in the synthesis of SCFAs, specifically enoyl-CoA hydratase, butyryl-CoA dehydrogenase, and electron transfer flavoproteins, and the ptb-buk gene from C. acetobutyricum, which encodes phosphotransbutyrylase and butyrate kinase. Additionally, key genes associated with competing metabolic pathways—ldhA, frdABCD, adhE, ackA, and pta—are deleted to optimize SCFA production, particularly butyrate. This strain is intended for use in therapeutic applications where enhanced SCFA production is beneficial, such as in the treatment of coronary heart disease.

### REFERENCE TO SEQUENCE DISCLOSURE

The sequence listing file under the file name “P2983US01_Sequence Listing.xml” submitted in ST.26 XML file format with a file size of 71 KB created on Oct. 14, 2024 and filed on Oct. 18, 2024 is incorporated herein by reference.

### CROSS-REFERENCE TO RELATED APPLICATIONS

The present application claims priority from U.S. provisional patent application Ser. No. 63/602,348 filed Nov. 22, 2023, and the disclosure of which is incorporated herein by reference in its entirety.

### FIELD OF THE INVENTION

The present invention generally relates to the field of biotechnology. More specifically the present invention relates to a genetically engineered probiotic strain.

### BACKGROUND OF THE INVENTION

Coronary heart disease (CHD), including myocardial infarction (MI), remains a leading cause of global mortality, responsible for over 800,000 deaths annually. The irreversible damage caused by MI significantly impairs cardiac contractile function, resulting in a five-year survival rate of only 49% following the onset of symptoms. Given the heart's extremely limited regenerative capacity, current therapeutic options, including surgical and pharmacological interventions, can merely delay the progression of this life-threatening condition. As such, prevention is recognized as the most effective strategy for reducing the incidence and mortality associated with CHD. In pursuit of this goal, various therapeutic approaches have been developed and explored; however, aside from lifestyle modifications, none have consistently demonstrated significant and reliable outcomes.

Emerging evidence underscores the pivotal role of the gut microbiome in maintaining health and modulating disease states by regulating homeostasis or producing beneficial compounds such as short-chain fatty acids (SCFAs). SCFAs, characterized by fatty acids with fewer than six carbon atoms, are essential nutrients for the caeco-colonic epithelium and are produced by the gut microbiota in the distal intestinal tract. The three most prevalent SCFAs, acetic acid, propionic acid, and butyric acid, are crucial end-products of colonic fermentation, derived from macronutrients like dietary fiber, resistant starches, and sugars or proteins that escape digestion in the upper intestinal tract. Recent studies have revealed that SCFAs positively influence cardiac health by inhibiting inflammation, balancing gene expression, and modulating immune cell activation. For instance, in a mouse model of hypertension, both a high-fiber diet and acetate supplementation significantly reduced blood pressure and mitigated cardiac and renal fibrosis. Furthermore, propionate treatment markedly attenuated hypertension, cardiac hypertrophy, fibrosis, and vascular dysfunction in two independent experimental models of hypertension and atherosclerosis. These findings provide compelling evidence that SCFAs play a protective role against adverse cardiac hypertrophy and the progression of fibrosis.

Despite the numerous health benefits associated with SCFAs, maintaining physiological concentrations of SCFAs in the plasma remains a significant challenge due to their rapid metabolism and utilization as an energy source throughout the body. Previous strategies, including intravenous injection, oral administration of water-dissolved or microencapsulated SCFAs, and the use of pro-drugs, have failed to achieve satisfactory results. Although the incorporation of fermentable fibers or chemically modified resistant starches into the diet has shown promise in delivering SCFAs, individual variations in gut microbiota composition have led to inconsistent outcomes.

Therefore, there is a pressing need for the development of effective and targeted delivery systems for SCFAs, and the present invention aims to address this critical challenge.

### SUMMARY OF THE INVENTION

It is an objective of the present invention to provide strains, or methods to solve the aforementioned technical problems.

In accordance with a first aspect of the present invention, a genetically engineered Escherichia coli Nissle 1917 (EcN) probiotic strain is provided. Specifically, the strain has a modified genome. The modified genome is modified to include an atoB gene, derived from E. Coli K-12, encoding acetyl-CoA acetyltransferase and having a sequence of SEQ ID NO: 01; a crt-bcd-etfA-etfB-BHBD gene cluster, derived from E. C. butyricum, encoding enoyl-CoA hydratase, butyryl-CoA dehydrogenase, alpha and beta subunits of electron transfer flavoprotein, and 3-hydroxybutyryl-CoA dehydrogenase and having a sequence of SEQ ID NO: 02; and a ptb-buk gene, derived from C. acetobutyricum, encoding phosphotransbutyrylase and butyrate kinase and having a sequence of SEQ ID NO: 03. It is worth noting that ldhA, frdABCD, adhE, ackA and pta genes are deleted from the modified genome.

In accordance with one embodiment of the present invention, the genetically engineered EcN probiotic strain constantly secretes SCFAs and elevates the blood concentration of the SCFAs in the subject.

In accordance with one embodiment of the present invention, the SCFAs comprise propionate and butyrate.

In accordance with one embodiment of the present invention, the deletion of the ldhA, frdABCD, adhE, ackA, and pta genes minimizes the production of competing metabolic byproducts, thereby increasing the efficiency of butyrate synthesis.

In accordance with one embodiment of the present invention, the competing metabolic byproducts includes lactate, ethanol, succinate, and acetate.

In accordance with one embodiment of the present invention, the ldhA gene has a sequence of SEQ ID NO: 04, the frdABCD gene has a sequence of SEQ ID NO: 05, the adhE gene has a sequence of SEQ ID NO: 06, the ackA gene has a sequence of SEQ ID NO: 07 and the pta gene has a sequence of SEQ ID NO: 08.

In accordance with a second aspect of the present invention, a composition for treating a coronary heart disease in a subject in need thereof. Particularly, the composition includes a genetically engineered EcN probiotic strain and a pharmaceutically acceptable carrier. The genetically engineered EcN probiotic strain has a modified genome, and the modified genome is modified to include an atoB gene, derived from E. Coli K-12, encoding acetyl-CoA acetyltransferase and having a sequence of SEQ ID NO: 01; a crt-bcd-etfA-etfB-BHBD gene cluster, derived from E. C. butyricum, encoding enoyl-CoA hydratase, butyryl-CoA dehydrogenase, alpha and beta subunits of electron transfer flavoprotein, and 3-hydroxybutyryl-CoA dehydrogenase and having a sequence of SEQ ID NO: 02; and a ptb-buk gene, derived from C. acetobutyricum, encoding phosphotransbutyrylase and butyrate kinase and having a sequence of SEQ ID NO: 03. Specifically, ldhA, frdABCD, adhE, ackA and pta genes are deleted from the modified genome. The genetically engineered EcN probiotic strain constantly secretes propionate and butyrate and elevates the blood concentration thereof in the subject.

In accordance with one embodiment of the present invention, the pharmaceutically acceptable carrier is selected from a diluent, a cryoprotectant, an encapsulation agent, a stabilizer, a buffer, an antioxidant, a preservative, a disintegrant, a binding agent, a coating agent, a liquid carrier, or a food-based carrier.

In accordance with one embodiment of the present invention, the composition further includes a dietary fiber source that enhances the production of propionate and butyrate by the genetically engineered EcN probiotic strain.

In accordance with one embodiment of the present invention, the genetically engineered EcN probiotic strain is encapsulated in a biocompatible polymer to improve its viability during storage and delivery.

In accordance with one embodiment of the present invention, the pharmaceutically acceptable carrier is formulated for oral administration in the form of a capsule, tablet, powder, or liquid suspension.

In accordance with one embodiment of the present invention, the ldhA gene has a sequence of SEQ ID NO: 04, the frdABCD gene has a sequence of SEQ ID NO: 05, the adhE gene has a sequence of 06, the ackA gene has a sequence of SEQ ID NO: 07 and the pta gene has a sequence of SEQ ID NO: 08.

In accordance with a third aspect of the present invention, a method of treating a coronary heart disease in a subject in need thereof is presented. The method includes orally administering a therapeutically effective amount of the aforementioned pharmaceutical composition to the subject.

In accordance with one embodiment of the present invention, the coronary heart disease is selected from myocardial infarction, atherosclerosis, angina pectoris, coronary artery disease, or ischemic heart disease.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the invention are described in more details hereinafter with reference to the drawings, in which:

FIGS. 1A-1D depict the generation of engineered EcN producing propionate and butyrate, in which FIG. 1A depicts the major metabolic pathways and metabolic engineering strategies employed to develop two engineered EcN TLP and TLB strains, FIG. 1B shows the measurement of SCFAs in the culture medium of EcN-WT, TLP, and TLB using GC-MS analysis, FIG. 1C shows the growth of EcN-WT, TLP, and TLB in BHI medium in aerobic conditions (37° C., shaking) and anaerobic conditions (37° C., anaerobic jar), and FIG. 1D displays the virulence assessment of the engineered E. coli in adhesion and invasion into intestinal Caco-2 cells;

FIGS. 2A-2B depict the map sequences of the plasmid pTLP (FIG. 2A) and pTLB (FIG. 2B), respectively;

FIGS. 3A-3D depict that EcN_TL formula improves SCFAs level in vivo, in which FIG. 3A shows the residence of bacteria in mouse gut after antibiotic pre-treatment followed by daily administration of EcN_EV, FIG. 3B demonstrates the SCFA concentrations in plasma and cecum, FIG. 3C depicts the residence of EcN_TL in mouse gut after antibiotic treatment followed by daily administration of EcN_TL, and FIG. 3D shows the plasma SCFAs concentration on mice fed with 7 doses of EcN_TL;

FIG. 4 depicts the SCFA concentrations in rat plasma;

FIGS. 5A-5D depict the effect of EcN_TL on improving cardiac function after myocardial infarction, in which FIG. 5A is a schematic of EcN_TL administration in animals, FIG. 5B shows the representative images M-mode of three groups at 1, 4 weeks post I/R and the measurement of left ventricular ejection fraction (EF), left fractional shortening (FS), left-ventricular internal diameter at end-diastole (LVIDd), left-ventricular internal diameter at end-systole (LVIDs), septal wall thickness (SWT), and posterior wall thickness (PWT), FIG. 5C displays the representative images of the hemodynamic pressure and volume (PV) curve on steady-state at 4 weeks post I/R injury and the measurement of cardiac output, stroke volume, volume max (V max) at end-diastole, maximal rate of pressure changes during systole (dP/dtmax), and minimal rate of pressure changes during diastole (dP/dtmin), and FIG. 5D shows the slope of end-systolic pressure volume relationship (ESPVR);

FIG. 6 depicts the serial echocardiography measurement data of individual rat until 4 weeks after myocardial infarction;

FIGS. 7A-7F depict the effect of EcN_TL on improving the microenvironment of the damaged heart, in which FIG. 7A shows the representative images of cardiomyocytes stained with cTnT (green) on the infarct zone and border zone at 4 weeks and the quantification, FIG. 7B depicts the representative images of capillaries stained with CD31 (red) on the infarct zone and border zone at 4 weeks and the quantification, FIG. 7C displays the representative images of Masson's trichrome staining at 4 weeks and quantification of a percentage of fibrosis and viable myocardium, FIG. 7D shows the representative images of de-natured collagen stained with CHP (red) and cardiomyocytes (green) on the infarct zone at 4 weeks and the quantification, FIG. 7E depicts the representative images of apoptotic CMs in infarct zone 3 day after myocardial infarction induction and quantification, and FIG. 7F shows the representative images of apoptotic ECs in infarct zone 3 day after myocardial infarction induction and quantification;

FIG. 8 depicts the measurement of immune cells in blood after MI;

FIGS. 9A-9C depict the inhibition of acute inflammatory reactions of EcN_TL, in which FIG. 9A depicts the representative images of neutrophils stained with MPO in the infarct zone 3 days after myocardial infarction induction and the quantification, FIG. 9B shows the representative images of MI macrophages in the infarct zone 3 days after myocardial infarction induction and the quantification, and FIG. 9C displays the representative images of M2 macrophages in infarct zone 3 days after myocardial infarction induction and quantification;

FIG. 10 depicts that SCFAs do not protect cardiac cells from ischemic stress;

FIGS. 11A-11H depict that SCFAs protect cardiomyocytes during inflammation, in which FIG. 11A and FIG. 11B show that the combine of SCFAs improves cardiomyocyte survival under inflammatory injury, FIG. 11C depicts the qPCR results of pro-inflammatory cytokine and anti-inflammatory cytokine gene expression in cardiomyocytes during inflammation triggered by LPS, FIG. 11D depicts that SCFA treatment inhibits phosphorylation of NF-κB and IκBα in cardiomyocytes, FIG. 11E shows that SCFA treatment inhibits phosphorylation of IKK α/β in cardiomyocytes, FIG. 11F shows that SCFA acts as NF-kB inhibitors, FIG. 11G depicts the immunocytochemistry of cardiomyocytes stained with MCT1, and FIG. 11H shows that an MCT1 inhibitor, syrosingopine, abolishes SCFAs protection effect; and

FIGS. 12A-12E depict the effect of SCFAs on innate immune cells, in which FIG. 12A shows that the pro-inflammatory cytokines TNFα and IL1β are downregulated in the presence of SCFAs under MI condition, FIG. 12B demonstrates that the anti-inflammatory cytokine IL1RA and wound-healing extracellular matrix FN are upregulated by SCFAs under M2 condition, FIG. 12C and FIG. 12D show that the cytokine array for supernatant in macrophage culture showing log 2 fold changes on secreted cytokines in M1 (FIG. 12C) and M2 (FIG. 12D) macrophages in the presence of SCFAs to that of M1 and M2 only controls without SCFAs, and FIG. 12E depicts that SCFAs inhibit neutrophil migration.

### DETAILED DESCRIPTION

In the following description, strains, compositions, and/or methods of treating coronary heart diseases and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

The term “crt-bcd-etfA-etfB-BHBD gene cluster” used herein refers to a gene cluster commonly found in certain anaerobic bacteria involved in the production of short-chain fatty acids, such as butyrate and butanol, through the fermentation of organic substrates.

The crt gene encodes the enzyme crotonase (also known as enoyl-CoA hydratase). This enzyme is involved in the metabolism of fatty acids and is a key enzyme in the butyrate synthesis pathway. It catalyzes the hydration of enoyl-CoA to hydroxyacyl-CoA, which is an essential step in converting intermediates into butyryl-CoA.

The bed gene encodes the enzyme butyryl-CoA dehydrogenase. This enzyme catalyzes the oxidation of butyryl-CoA to crotonyl-CoA in the process of fatty acid metabolism and energy production, particularly in the butyrate synthesis pathway.

The etfA and etfB genes encode for the alpha and beta subunits of electron transfer flavoprotein (ETF). ETFs are crucial in transferring electrons from primary dehydrogenases (such as butyryl-CoA dehydrogenase) to the respiratory chain, helping in energy production through the generation of ATP.

The BHBD gene encodes for the enzyme 3-hydroxybutyryl-CoA dehydrogenase, also known as β-hydroxybutyryl-CoA dehydrogenase. This enzyme is involved in the conversion of 3-hydroxybutyryl-CoA to acetoacetyl-CoA, a key intermediate in the synthesis of butyrate.

These genes work together in the butyrate synthesis pathway: crt and BHBD are involved in the conversion of intermediates during fatty acid metabolism; bcd catalyzes the key dehydrogenation step in butyrate synthesis; and etfA and etfB facilitate electron transfer during these metabolic processes, supporting energy production. This gene cluster is essential in producing butyrate, a short-chain fatty acid that plays a significant role in gut health and energy metabolism.

Due to the extremely limited regenerative potential, heart disease-related injuries inevitably result irreversible and fatal damage to the heart. Consequently, apart from making lifestyle changes, the most promising approach to safeguard the heart from injury is through effective prevention strategies. However, currently available preventative strategies are largely inadequate and mostly ineffective. E. coli Nissle 1917 strain (EcN) is a short-lived probiotic isolated from the human gut and has been widely used for over a century to treat inflammatory bowel disease and irritable bowel syndrome. Its long history of safety, tolerability in the human body, knowledge of its molecular background, and availability of genetic manipulation tools make EcN an ideal candidate for the development of engineered therapies.

Therefore, in the present invention, a novel strategy is successfully developed to prevent ischemic heart disease by creating an engineered probiotic. This probiotic continuously and consistently secretes a physiological level of SCFAs into the bloodstream. This engineered probiotic may be orally administered as part of a preventative program for ischemic heart disease.

In accordance with a first aspect of the present invention, a genetically engineered EcN probiotic strain is provided. The genetically engineered EcN probiotic strain is modified to include specific genetic sequences aimed at enhancing its ability to produce SCFAs, particularly propionate and butyrate, which have significant therapeutic potential. The engineered strain has been designed with a particular focus on optimizing metabolic pathways to maximize the production of these SCFAs, which are known for their health-promoting effects, particularly in relation to cardiovascular diseases.

In the modified genome of the EcN strain, several key genes have been incorporated to enable the biosynthesis of SCFAs. Firstly, the atoB gene, derived from E. coli K-12, has been inserted. This gene encodes the enzyme acetyl-CoA acetyltransferase, which is critical for the conversion of acetyl-CoA into acetoacetyl-CoA, an essential step in the production of SCFAs. The specific sequence of this gene is represented by SEQ ID NO: 01.

Additionally, the strain includes a gene cluster known as crt-bcd-etfA-etfB-BHBD, which is derived from E. C. butyricum. This cluster encodes a series of enzymes including enoyl-CoA hydratase, butyryl-CoA dehydrogenase, the alpha and beta subunits of electron transfer flavoprotein, and 3-hydroxybutyryl-CoA dehydrogenase. These enzymes work in concert to facilitate the production of butyrate, an SCFA with potent anti-inflammatory properties. The sequence of this gene cluster is detailed in SEQ ID NO: 02.

To further enhance the production of butyrate, the ptb-buk gene from C. acetobutyricum has been introduced into the EcN strain. This gene encodes the enzymes phosphotransbutyrylase and butyrate kinase, which are pivotal in the conversion of butyryl-CoA to butyrate. The specific sequence of the ptb-buk gene is provided as SEQ ID NO: 03.

In conjunction with the introduction of these genes, the genetically engineered EcN strain has also undergone a series of gene deletions to optimize its metabolic pathways for SCFA production. Specifically, the ldhA, frdABCD, adhE, ackA, and pta genes have been deleted. These deletions are strategically implemented to minimize the production of competing metabolic byproducts such as lactate, ethanol, succinate, and acetate, which could otherwise detract from the efficiency of butyrate synthesis. The sequences of the deleted genes are represented as SEQ ID NOs: 04, 05, 06, 07, and 08, respectively.

The resultant genetically engineered EcN probiotic strain is capable of continuously secreting SCFAs, thereby elevating the blood concentration of these beneficial fatty acids in the subject. This engineered strain holds significant promise for therapeutic applications, particularly in the management of coronary heart disease and other conditions where modulation of inflammation and metabolic health are crucial. The inclusion of specific gene sequences, coupled with targeted deletions, underscores the precision and effectiveness of this engineered probiotic strain in promoting health through enhanced SCFA production.

In accordance with a second aspect of the present invention, a composition designed for the treatment of coronary heart disease (CHD) in subjects who require such intervention is introduced. This composition includes a genetically engineered EcN probiotic strain, which has been meticulously modified to possess a unique genetic configuration aimed at enhancing its therapeutic potential. The engineered strain, combined with a pharmaceutically acceptable carrier, offers a novel approach to managing CHD by leveraging the production of specific SCFAs, particularly propionate and butyrate, which have demonstrated cardioprotective effects.

The genetically engineered EcN probiotic strain included in the composition is characterized by a genome that has been enhanced with several key genetic elements. Specifically, the genome comprises an atoB gene, derived from E. coli K-12, which encodes the enzyme acetyl-CoA acetyltransferase. This enzyme plays a crucial role in the metabolic pathway that leads to the synthesis of SCFAs. The sequence of the atoB gene is provided as SEQ ID NO: 01.

In addition to the atoB gene, the genome of the engineered EcN strain includes a crt-bcd-etfA-etfB-BHBD gene cluster, derived from E. C. butyricum. This gene cluster encodes a series of enzymes essential for butyrate synthesis, including enoyl-CoA hydratase, butyryl-CoA dehydrogenase, the alpha and beta subunits of electron transfer flavoprotein, and 3-hydroxybutyryl-CoA dehydrogenase. The inclusion of this gene cluster ensures the efficient production of butyrate, a SCFA known for its anti-inflammatory and cardioprotective properties. The sequence of this gene cluster is identified as SEQ ID NO: 02.

Further enhancing the strain's ability to produce SCFAs, the genome incorporates the ptb-buk gene, derived from C. acetobutyricum. This gene encodes the enzymes phosphotransbutyrylase and butyrate kinase, which are critical for the conversion of butyryl-CoA to butyrate, thereby boosting the strain's butyrate output. The sequence of the ptb-buk gene is detailed as SEQ ID NO: 03.

To optimize the strain's metabolic efficiency and to focus its activity on SCFA production, specific genes have been strategically deleted from its genome. These deleted genes include ldhA, frdABCD, adhE, ackA, and pta. The deletion of these genes reduces the production of competing metabolic byproducts, such as lactate, ethanol, succinate, and acetate, which could otherwise interfere with the synthesis of SCFAs like butyrate. The sequences of the deleted genes are provided as SEQ ID NO: 04, SEQ ID NO: 05, SEQ ID NO: 06, SEQ ID NO: 07, and SEQ ID NO: 08, respectively.

The genetically engineered EcN probiotic strain within the composition is capable of constantly secreting propionate and butyrate, which leads to an elevation in the blood concentrations of these SCFAs in the subject. This continuous secretion is critical for the therapeutic effectiveness of the composition, as these SCFAs have been shown to exert significant benefits in reducing inflammation, modulating immune responses, and protecting against the progression of CHD.

The composition also includes a pharmaceutically acceptable carrier, which is selected from a wide range of substances that are suitable for use in pharmaceutical formulations. These carriers may include diluents, cryoprotectants, encapsulation agents, stabilizers, buffers, antioxidants, preservatives, disintegrants, binding agents, coating agents, liquid carriers, or food-based carriers. The choice of carrier depends on the intended formulation and delivery method of the composition.

To further enhance the production of propionate and butyrate by the genetically engineered EcN strain, the composition may also comprise a dietary fiber source. This fiber source serves as a substrate for the microbial fermentation process, thereby boosting the production of these beneficial SCFAs within the gut.

In some embodiments, the genetically engineered EcN probiotic strain may be encapsulated in a biocompatible polymer. This encapsulation improves the viability of the strain during storage and delivery, ensuring that the probiotic remains effective when administered to the subject.

The composition is formulated for oral administration, which can be provided in various forms such as capsules, tablets, powders, or liquid suspensions. These forms are designed to facilitate case of administration and ensure the delivery of the probiotic strain and its SCFA products to the appropriate site of action within the gastrointestinal tract.

Overall, this detailed description outlines a comprehensive approach to treating coronary heart disease through the use of a genetically engineered probiotic strain that has been specifically modified to optimize the production of cardioprotective SCFAs, combined with a suitable pharmaceutical carrier and, optionally, additional components to enhance efficacy.

In accordance with a third aspect of the present invention, a method for treating CHD in subjects who require such therapeutic intervention is presented. This method involves the oral administration of the aforementioned pharmaceutical composition, having the genetically engineered EcN strain. The genetically engineered EcN strain is specifically designed to produce SCFAs that are known to have beneficial effects on cardiovascular health, particularly in the context of coronary heart disease.

In the method, the coronary heart disease being treated can be any one of several conditions associated with the heart and coronary arteries. These conditions include, but are not limited to, myocardial infarction, atherosclerosis, angina pectoris, coronary artery disease, or ischemic heart disease. By administering the genetically engineered EcN strain in the form of the described pharmaceutical composition, the method aims to alleviate symptoms, slow disease progression, and improve overall cardiovascular health in subjects suffering from these coronary conditions.

The therapeutic approach provided by this method represents a novel use of genetically engineered probiotics in the management of coronary heart disease. By leveraging the strain's ability to produce cardioprotective SCFAs within the gastrointestinal tract, the method offers a promising alternative or adjunct to traditional treatments for heart disease, potentially improving outcomes for patients suffering from these life-threatening conditions.

### EXAMPLES

### Example 1. Genetically Modified Probiotics Efficiently Produce SCFAs

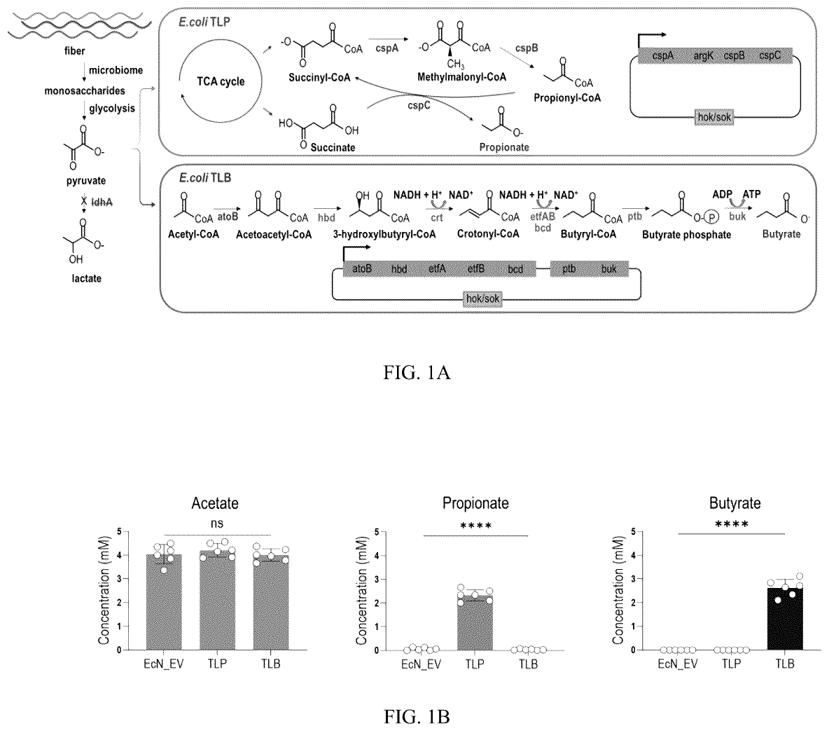

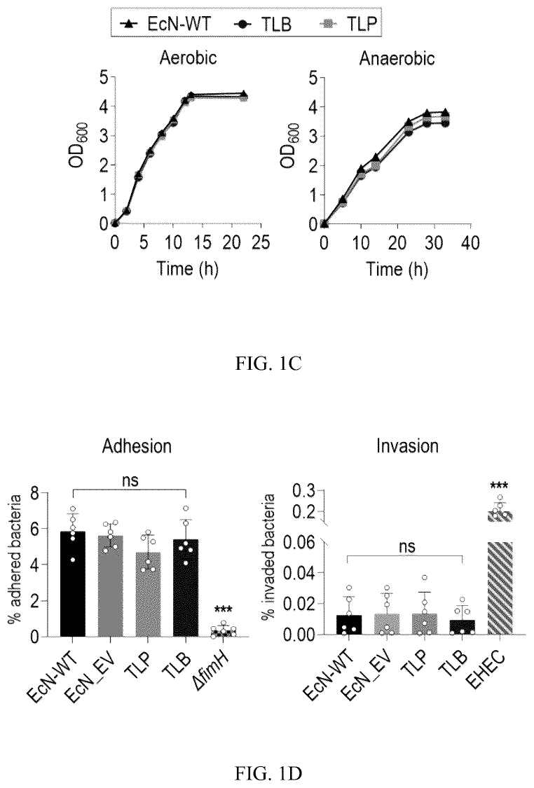

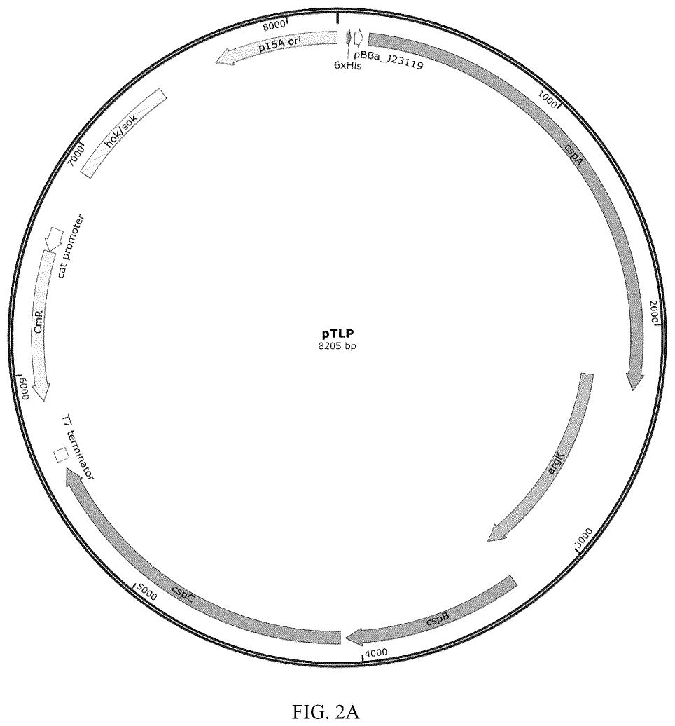

Wildtype Escherichia coli Nissle 1917 (EcN) does not efficiently produce propionate and butyrate. Therefore, separate biosynthesis pathways for propionate and butyrate are introduced into the bacteria. The genetic cassette of the sleeping beauty mutase (sbm), which is responsible for propionate biosynthesis and carries the cspA, cspB, cspC, and argK genes from commensal E. coli K-12, is cloned into the pTLP plasmid. This plasmid was then transformed into EcN to enhance propionate production. For butyrate production, a heterologous pathway was introduced into the bacteria using the pTLB plasmid. This plasmid contained atoB from E. coli K-12, BHBD, crt, etfAB, and bcd from Clostridium butyricum, and ptb and buk from Clostridium acetobutyricum (FIG. 1A). The genes cloned from C. butyricum, and C. acetobutyricum have previously been demonstrated to efficiently produce butyrate in E. coli. Since pyruvate is the precursor of both pathways, the pTLP and pTLB plasmids are introduced separately into EcN to prevent competition for pyruvate within a single strain to guarantee the efficient production of propionate and butyrate. Additionally, the hok/sok system is also included to maintain the plasmids in the bacteria.

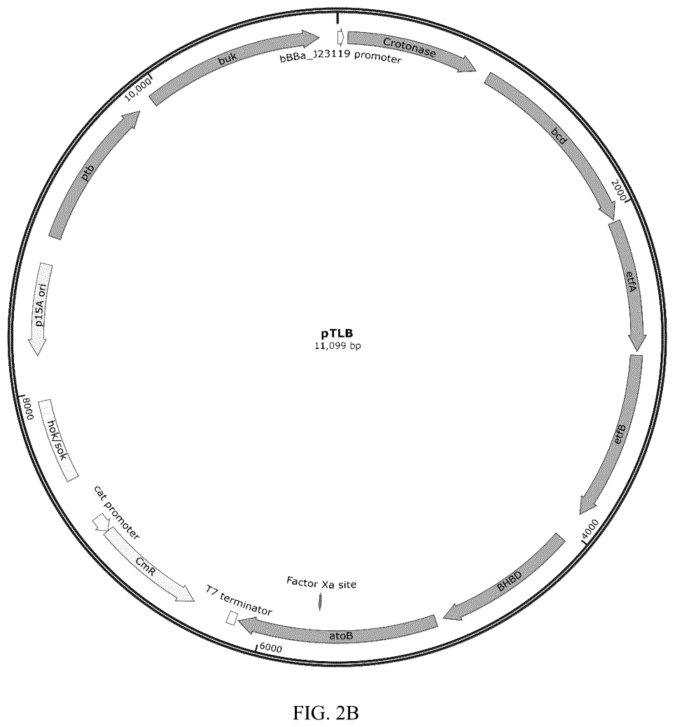

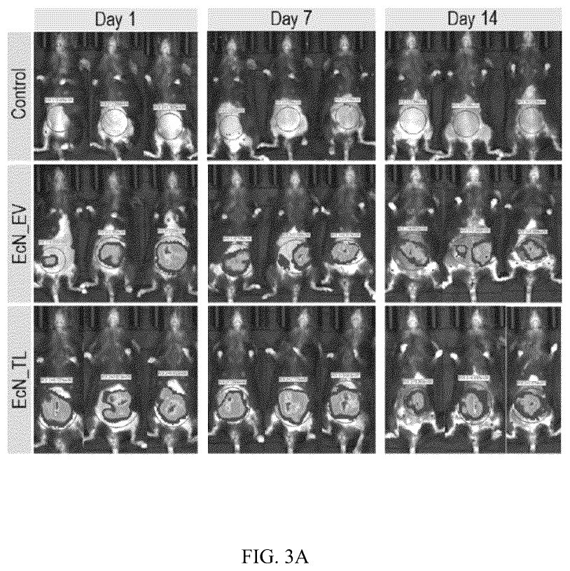

The plasmid pQH is generated by removing lacI gene and adding hok/sok system into plasmid pACYCT2, which served as the backbone. For the construction of pTLP, the sleeping beauty mutase (sbm) operon is amplified from E. coli K-12 via PCR and cloned into pQH plasmid using the CloneExpress Ultra One Step Cloning Kit (Vazyme). Similarly, for the construction of pTLB, the genes atoB (SEQ ID NO: 01), crt-bcd-etfA-etfB-BHBD (SEQ ID NO: 02) and ptb-buk (SEQ ID NO: 03) are amplified from E. coli K-12, C. butyricum and C. acetobutyricum, respectively. The genes are then arranged in an order on the pQH plasmid. The butyrate-production cassette is arranged in the order: crt-bcd-etfA-etfB-BHBD, placed under a strong constitutive promoter, pBBa_J23119. The ptb-buk (SEQ ID NO: 03) are expressed under its native promoter, placed upstream the crt-bcd-etfA-etfB-BHBD cassette (SEQ ID NO: 02) on plasmid pTLB. The propionate-production cassette sbm is placed under promoter pbBBa_J23119 on plasmid pTLP. The maps of the two plasmids, pTLP and pTLB are depicted in FIGS. 2A and 2B.

To optimize SCFA production by channeling carbon flux into the designated pathways, various competing genes at the pyruvate and acetyl-CoA nodes are knocked out from the EcN genome, including ldhA (encoding lactate dehydrogenase, SEQ ID NO: 04), frdABCD (encoding fumarate reductase enzyme complex, SEQ ID NO: 05), adhE (encoding aldehyde-alcohol dehydrogenase, SEQ ID NO: 06), ackA (encoding acetate kinase, SEQ ID NO: 07), and pta (encoding phosphate acetyltransferase, SEQ ID NO: 08). As shown in FIG. 1B, the null mutant of ldhA, in the presence of either TLP or TLB plasmids, produces a significant amount of propionate (EcN: ΔldhA/pTLP: 2.5±0.17 mM) and butyrate (EcN: ΔldhA/pTLB: 2.87±0.24 mM) without affecting growth under both aerobic and anaerobic conditions (FIG. 1C). In contrast, other null mutants such as ΔadhE, Δfrd, ΔackA, and Δpta show reduced anaerobic growth, rendering them unsuitable for in vivo studies. To ensure the safety of these genetically modified bacteria, their virulence is compared to that of wild-type EcN using an in vitro Caco-2 cell model (intestine epithelial cells). As shown in FIG. 1D, no significant difference in adhesion and invasion is observed between wildtype and the engineered probiotics. To simplify the nomenclature of the different strains, EcN: ΔldhA/pTLP and EcN: ΔldhA/pTLB are designated as TLP and TLB, respectively. Moreover, a 1:1 mass ratio mixture of TLP and TLB, named EcN_TL, is used for studying the effect of SCFAs in the animal models. EcN transduced with empty vector (EcN_EV) is used as a control.

The gene deletion in EcN is performed using the lambda Red system, as described previously. Briefly, the EcN containing plasmid pSIJ8-Cam^{R} is cultured at 30° C. Induction of the λ Red system is achieved by adding arabinose to a final concentration of 15 mM and incubating for 30 minutes. The PCR product of the Kanamycin-resistant cassette, with 50 bp (40 bp with pta gene) homolog to the target genes, is then electroporated into EcN/pSIJ8-Cam^{R} and allowed to recover overnight. The recombinants are selected on agar plates containing Kanamycin at a concentration of 50 μg/mL. Confirmation of target gene disruption is performed through PCR and sequencing of the target genes. The primer sequences used for plasmid construction and gene disruption are shown in Table 1.

TABLE 1
The oligonucleotide sequences

| Name | Sequence (5′-3′) |
|---|---|
| hok/sok forward primer | ATCGGCTAGCTGATGCGGCAACAATCACAC (SEQ ID NO: |
| | 09) |
| hok/sok reverse primer | TGCAGCTAGCAGTCAGACCAGCATCAGTCC (SEQ ID NO: |
| | 10) |
| sbm forward primer | TAGAGTTTAAGGAGATATACATATGTCTAACGTGCAGGAGTG |
| | GCAAC (SEQ ID NO: 11) |
| sbm reverse primer | CAGCGGTGGCAGCAGCCTAGGTTAATTAACCCAGCATCGAGC |
| | CGGTTG (SEQ ID NO: 12) |
| atoB forward primer | AATATCCCGTTAAATAAATATAGGAGGTTAAGTAATGAAAAA |
| | TTGTGTCATCGTC (SEQ ID NO: 13) |
| atoB reverse primer | GCTAGTTATTGCTCAGCGGTTTAATTCAATCGTTCAATCACC |
| | (SEQ ID NO: 14) |
| BHBD forward primer | GCTTCTAGGAGTATATTTATTTAAC (SEQ ID NO: 15) |
| BHBD reverse primer | ACCGCTGAGCAATAAC (SEQ ID NO: 16) |
| ptb-delete forward | ATTAATCAGATAAAATATTTATTTTCAGAAAATTTAGCATTT |
| primer | AAAG (SEQ ID NO: 17) |
| ptb-delete reverse | ACTGAGCTAGCTGTAAAGAACATTTTTATAAATTCCATTTTT |
| primer | TCC (SEQ ID NO: 18) |
| ldhA-delete forward | ATGAAACTCGCCGTTTATAGCACAAAACAGTACGACAAGAAG |
| primer | TACCTGCAAATATCCTCCTTAGTTCCTATTCCG (SEQ ID |
| | NO: 19) |
| ldhA-delete reverse | TTAAACCAGTTCGTTCGGGCAGGTTTCGCCTTTTTCCAGATT |
| primer | GCTTAAGTCTGCTTCGAAGTTCCTATACTTTC (SEQ ID |
| | NO: 20) |
| adhE-delete forward | ATGGCTGTTACTAATGTCGCTGAACTTAACGCACTCGTAGAG |
| primer | CGTGTAAAAATATCCTCCTTAGTTCCTATTCCG (SEQ ID |
| | NO: 21) |
| adhE-delete reverse | TTAAGCGGATTTTTTCGCTTTTTTCTCAGCTTTAGCCGGAGC |
| primer | AGCTTCTTCTGCTTCGAAGTTCCTATACTTTC (SEQ ID |
| | NO: 22) |
| pckA-delete forward | TGTCAAATATGAATTTCTCCAGATACGTAAATCTATGAGCGA |
| primer | ACTTCAGAGCGCTTTTG (SEQ ID NO: 23) |
| pckA-delete reverse | AATATGTATTGCCTGAATAGTAAAGTCTTTTTGGGGGTGTGG |
| primer | TCACAGCTTGTCTGTAAG (SEQ ID NO: 24) |
| frdA-delete forward | TAAAAAAAGCACGATCTGATGGTTTAGTAATTAAATTAATCA |
| primer | TCTTCAGTAATATCCTCCTTAGTTCCTATTCCG (SEQ ID |
| | NO: 25) |
| frdA-delete reverse | GTTGCGTCATAAGGCACTTCATAGAATGCGCTATGCGGTGCG |
| primer | GTATCGACCTGCTTCGAAGTTCCTATACTTTC (SEQ ID |
| | NO: 26) |
| pta-delete forward | TGTAACCCGCCAAATCGGCGGTAACGAAAGAGGATAAACCGT |
| primer | GTCCCGTATTATTATGCTG (SEQ ID NO: 27) |
| pta-delete reverse | TTCAGATATCCGCAGCGCAAAGCTGCGGATGATGACGAGATT |
| primer | ACTGCTGCTGTGCAG (SEQ ID NO: 28) |
| r-IL 1b forward primer | AGAAGAGCCCGTCCTCTGTGA (SEQ ID NO: 29) |
| r-IL 1b reverse primer | TCAGACAGCACGAGGCATTT (SEQ ID NO: 30) |
| r-TNF forward primer | GCATGATCCGAGATGTGGAA (SEQ ID NO: 31) |
| r-TNF reverse primer | CAGACACCGCCTGGAGTTCT (SEQ ID NO: 32) |
| r-IL10 forward primer | GAATTCCCTGGGAGAGAAGC (SEQ ID NO: 33) |
| r-IL10 reverse primer | CGGGTGGTTCAATTTTTCAT (SEQ ID NO: 34) |
| r-IL6 forward primer | CACTTCACAAGTCGGAGGCT (SEQ ID NO: 35) |
| r-IL6 reverse primer | TCTGACAGTGCATCATCGCT (SEQ ID NO: 36) |
| h-TNFα forward primer | TCCCCAGGGACCTCTCTCTA (SEQ ID NO: 37) |
| h-TNFα reverse primer | GGGTTTGCTACAACATGGGCTA (SEQ ID NO: 38) |
| h-IL1β forward primer | ATGATGGCTTATTACAGTGGCAA (SEQ ID NO: 39) |
| h-IL1β reverse primer | GTCGGAGATTCGTAGCTGGA (SEQ ID NO: 40) |
| h-IL1RA forward primer | GAAGATGTGCCTGTCCTGTGT (SEQ ID NO: 41) |
| h-IL1RA reverse primer | CGCTCAGGTCAGTGATGTTAA (SEQ ID NO: 42) |
| h-FN forward primer | GAGAATAAGCTGTACCATCGCAA (SEQ ID NO: 43) |
| h-FN reverse primer | CGACCACATAGGAAGTCCCAG (SEQ ID NO: 44) |

### Example 2. Administration of the Engineered EcN Enhances Plasma SCFAs Level

The colonization of EcN_TL and subsequent secretion of SCFA in vivo are assessed. The luminescence cassette LuxCDABE from Photorhabdus luminescens is introduced to both EcN_TL and EcN_EV to allow in vivo monitoring of bacterial colonization in the gut using the in vivo imaging system (IVIS). To overcome the resistance of human-origin probiotics in the mouse gut, mice are pre-treated by an antibiotic cocktail (ABX). Briefly, eight-week-old C57BL/6J mice are randomly assigned to different groups. The mice are pre-treated with an antibiotic cocktail in their drinking water. The antibiotic cocktail consisted of vancomycin 0.125 g/L, neomycin 0.25 g/L, ampicillin 0.25 g/L, metronidazole 0.25 g/L, and 1% sucrose and is administered in the drinking water for a duration of one week. Prior to oral gavage, the mice are anesthetized with 2% inhaled isoflurane. A suspension of 109 colony-forming units (cfu) of bacteria in 200 μL PBS is then administered through the esophagus using an 18-gauge stainless steel feeding needle with a 2.25-mm ball (THOMAS, USA). The luminescent signal of the bacteria in the abdominal region is monitored daily using the Spectrum In Vivo Imaging System (Perkin Elmer). Mice are observed for at least 15 min immediately after oral gavage. The mice study is approved by the Institutional Animal Care and Use Committee (IACUC) of The Catholic University of Korea (Approval number: CUMC-2021-0243-06). All animal procedures conform to the NIH guidelines, or the guidelines issued by Directive 2010/63/EU of the European Parliament for the protection of animals used in scientific research. Eight-week-old C57BL/6J mice are randomly assigned to different groups.

At the end of the experiment, mice are sacrificed by inhaling 5% isoflurane. Subsequently, blood samples are collected from the right ventricle, and the cecum is also collected. The culture media are collected by centrifugation to remove bacteria cells and then snap-frozen in liquid nitrogen. Blood serum and cecal samples are collected immediately after sacrificing the animals and stored at −80° C. until analysis. The cecal content is homogenized with 1 ml of water and centrifuged at 12,000 rpm for 10 min at 4° C.

The blood samples are then analyzed for short-chain fatty acid levels using gas chromatography (GC)-mass spectrometry (MS). Briefly, 20 μL of supernatant or serum is derivatized by pentafluorobenzyl bromide (PFBBr, Sigma) in acetone at 60° C. for 30 min. The derivatized product is then extracted using a solvent extraction method with hexane and water. GC-MS analysis is performed using the Agilent 6890N GC coupled with 5975 inert Performance Turbo MSD (Agilent Technologies) in the selected ion monitoring (SIM) mode. SCFAs are identified by comparing retention time of sample peaks with external standards such as acetic acid, propionic acid, and butyric acid (Sigma). An external standard calibration is established to calculate the concentration of the sample.

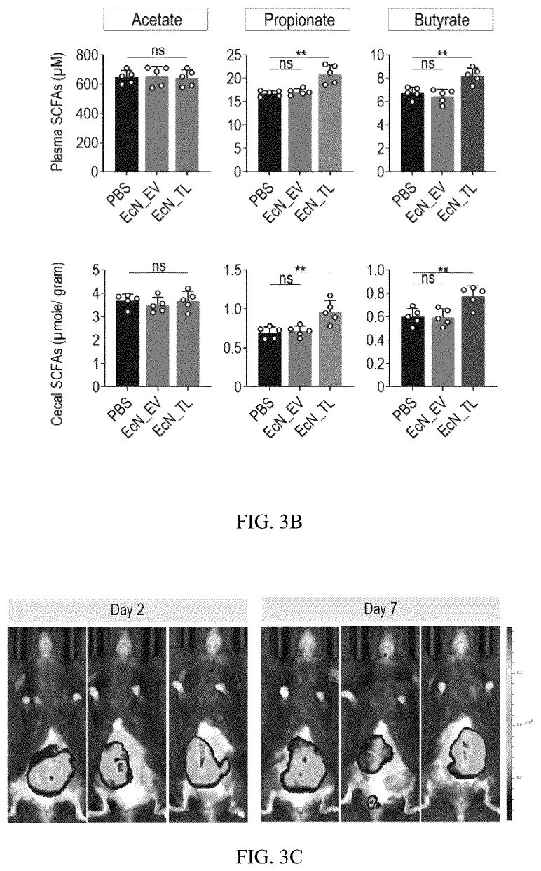

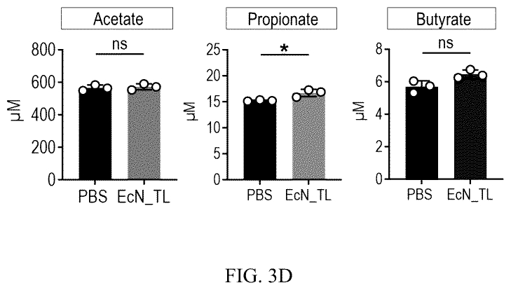

The results show that daily feeding of EcN_TL and EcN_EV for 14 days exhibits stable colonization of bacteria in the gut (FIG. 3A). The concentration of in the plasma is further measured. The plasma propionate concentration in the EcN_TL fed group is 24.66% higher than that in the PBS-fed and EcN_EV-treated groups (p<0.01). Similarly, EcN_TL improves butyrate concentration in the plasma by 22% compared to other treatment groups (p<0.01) (FIG. 3B). This enhancement is maintained after cessation of treatment, as the measurement is taken 24 hours after the last dose of probiotics. It is worth noting that seven days of daily feeding with EcN_TL only increases the propionate plasma concentration but not butyrate (FIG. 3C and FIG. 3D).

The probiotic regimen in rats in order to investigate the preventive effects of EcN_TL against myocardial I/R injury in the well-established rat model. The rat experiments are approved by the Institutional Animal Care and Use Committee (IACUC) of The Catholic University of Korea (Approval number: CUMC-2020-0051-01). All animal procedures conform to the NIH guidelines, or the guidelines issued by Directive 2010/63/EU of the European Parliament for the protection of animals used in scientific research. Briefly, Fisher 344 rats (160 to 180 g, 8-week-old males, Koatec, Korea) are given an antibiotic cocktail comprising vancomycin (0.125 g/L), neomycin (0.25 g/L), ampicillin (0.25 g/L), and metronidazole (0.25 g/L) in their drinking water for a week, similar to the mouse experiment. Afterward, the rats are administered 1011 cfu of probiotics in 1 mL PBS through the esophagus using the 18-gauge stainless steel feeding needle. The probiotics are given to the rats daily for 7 days before and after the surgery. For performing Ischemic/reperfusion injury model, the rats are anesthetized with 2% inhaled isoflurane and intubated with an 18G intravenous catheter. The rats are ventilated with a rodent respirator (Harvard Apparatus), and a 37° C. heating pad is used to maintain their body temperature throughout the operation. The chest is shaved and sterilized with 70% alcohol. Ischemia-reperfusion is induced temporarily by occluding the LAD artery with a 7-0 Prolene suture for 1 hour. The chest is aseptically closed and disinfected after surgery. To establish the baseline LV function, EF, and FS are examined 4 hours after surgery.

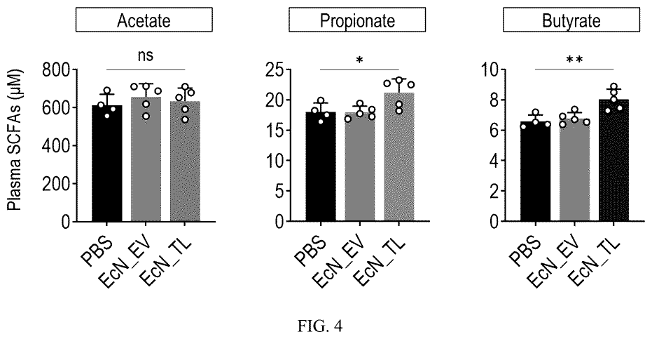

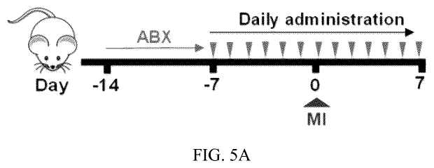

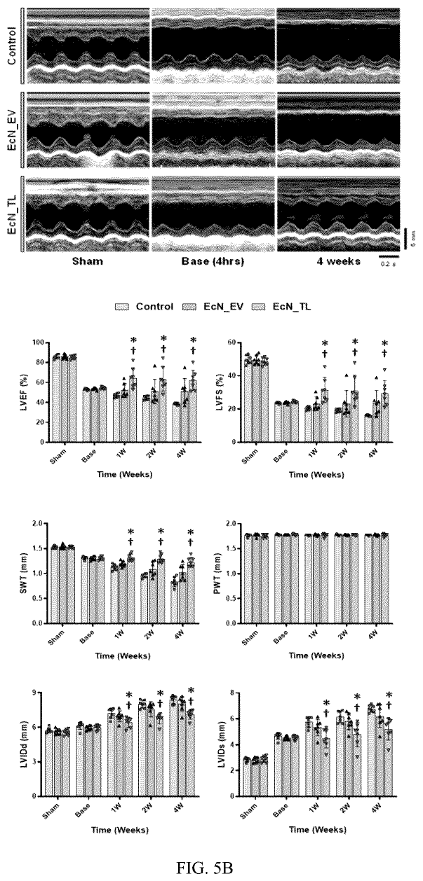

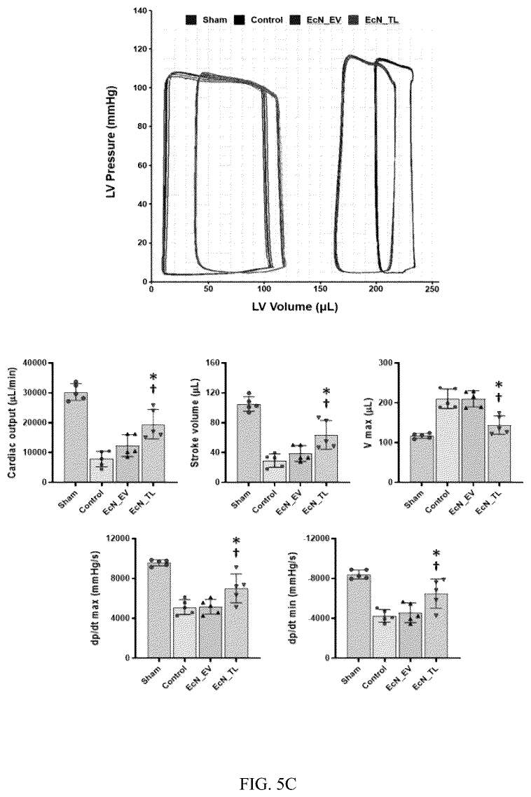

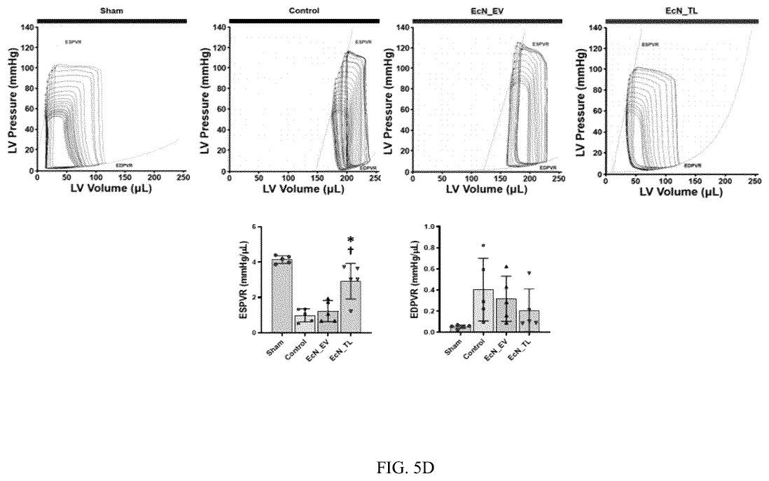

As shown in FIG. 4, daily oral administration of phosphate-buffered saline (PBS), EcN_EV, or EcN_TL for 14 days results in a similar enhancement of SCFA concentration in the blood of the rats. After the initial 7 days of daily oral administration, on day 7, I/R injury is induced by ligating the left anterior descending (LAD) coronary artery. Subsequently, the animals received an additional 7 daily doses of the treatment (FIG. 5A).

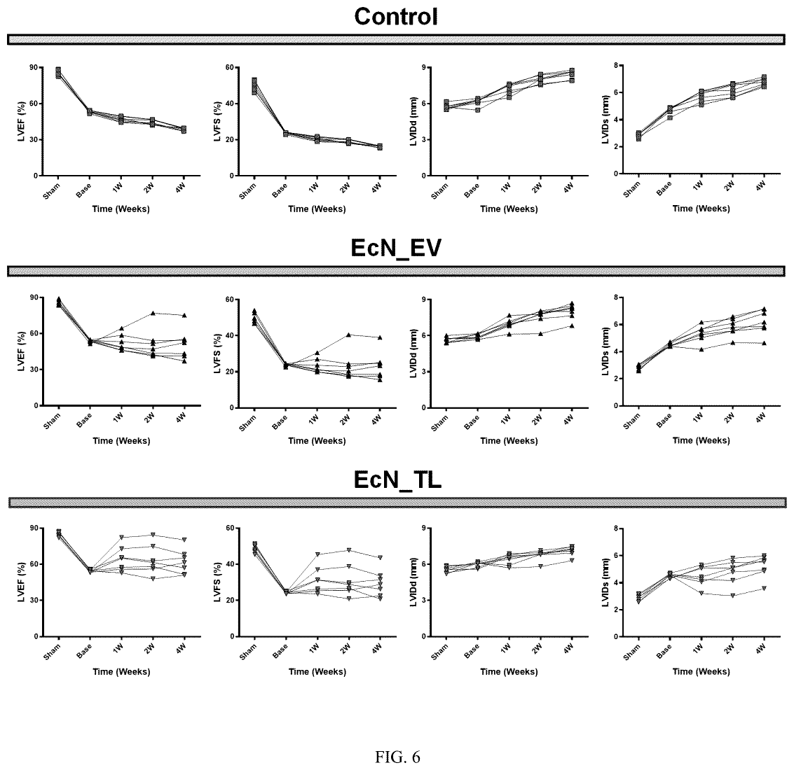

Serial echocardiography is performed at baseline, 1 week, 2 weeks, and 4 weeks after induction of I/R injury to evaluate the protective effect of EcN_TL. Briefly, the animals are anesthetized with 2% isoflurane and placed on a heating pad to maintain the body temperature at 37° C. Serial echocardiography was performed baseline (4 hours) and at 1, 2, and 4 weeks after the treatment using a transthoracic echocardiography system equipped with a 15 MHz L15-7io linear transducer (Affniti 50G, Philips) to determine the ejection fraction (EF), fractional shortening (FS), left-ventricular internal diameter at end-diastole (LVIDd), left ventricular internal diameter at end-systole (LVIDs), septal wall thickness (SWT), and posterior wall thickness (PWT). M-mode images show that EcN_TL remarkably enhances contractile activity and thickness in the left anterior wall after I/R, as compared to both the PBS-fed groups (control) and EcN_EV groups (FIG. 5B and FIG. 6). EcN_TL promotes EF and FS by 64.6% and 31.5%, respectively, at 1-week post-I/R (p<0.05). Additionally, EcN_TL groups exhibit lower left ventricular internal diastolic dimension and left ventricular internal systolic dimension (p<0.05), as well as higher septal wall thickness compared to the other experimental groups (p<0.05) (FIG. 5B).

Furthermore, pressure-volume (PV) loop analysis is conducted to directly assess cardiac function. Briefly, hemodynamic measurements are performed at the endpoint of 8 weeks before euthanasia. Rats are anesthetized and ventilated as described previously. After thoracotomy without bleeding, the LV apex of the heart is punctured with a 26 G needle and the 2F conductance catheter (SPR-838, Millar) is inserted into the LV. LV pressure-volume parameters are continually recorded using a PV conductance system (MPVS Ultra, emka TECHNOLOGIES, Paris, France) coupled to a digital converter (PowerLab 16/35, ADInstruments, Colorado Springs, CO). Load-independent parameters of cardiac function including the slopes of end systolic pressure volume relationship (ESPVR) and end-diastolic pressure volume relationship (EDPVR) are measured at different preloads, which are elicited by transient occlusion of the inferior vena cava with needle holder. The 50 μl of hypertonic saline (20% 22 NaCl) is injected into the left jugular vein to calculate the parallel conductance after hemodynamic measurements. The blood is collected from the left ventricle into a heparinized syringe and transferred into cuvettes to convert the conductance signal to volume using the catheter. The absolute volume of the rat is defined by calibrating the parallel conductance and the cuvette conductance.

Hemodynamic criteria such as cardiac output, cardiac stroke, Vmax, and dP/dt max/min demonstrate significant enhancement of left ventricular function in the EcN_TL groups (p<0.05) (FIG. 5C). The EcN_TL-fed rats also exhibit a 1.9-fold higher end-systolic pressure-volume relationship (ESPVR) and a trend of reduction in the slope of the end-diastolic pressure-volume relationship (EDPVR) compared to both control and EcN_EV-fed groups (p<0.05), indicating improved intrinsic cardiac contractibility as it represents the maximal pressure generated by the left ventricle at any given volume (FIG. 5D). Collectively, these results clearly indicate that pre-treatment with EcN_TL protects the heart from myocardial I/R injury, significantly improves heart function, and attenuated adverse left ventricle remodeling following I/R injury.

Next, the morphological and pathological changes in the hearts of the control, EcN_EV, and EcN_TL groups are examined. The heart sections are incubated in blocking solution for 1 h at room temperature and then incubated at 4° C. overnight with one of the following antibodies: goat anti-CD31 (1:200; R&D Cat #AF3628), rabbit anti-MPO (1:50; Abcam Cat #ab9535), mouse anti-CD68 (1:100; Abcam Cat #ab955), mouse anti-iNOS (1:200; Abcam Cat #ab49999), rabbit anti-CD206 (1:100; Abcam Cat #ab64693), and mouse anti-cTnT (1:200; Abcam Cat #ab8295). Confocal images are captured at room temperature with ZEN software on an upright confocal microscope with the predefined ZEN software configurations for Alexa Fluor 546, Alexa Fluor 488, and DAPI. Additionally, a terminal deoxynucleotidyl-transferase-mediated dUTP nick end-labeling kit (Roche; Cat #11684795910) assay is used to identify apoptosis. The sections are deparaffinized with xylene and rehydration. Then, the sections are permeabilized with 200 μl of TBS-T for 2 min on ice. The sections are incubated in a staining solution containing deoxynucleotidyl transferase for 1 h at 37° C. in the dark. After washing with PBS, the sections are mounted with DAPI mounting solution (Vector; H-1500)

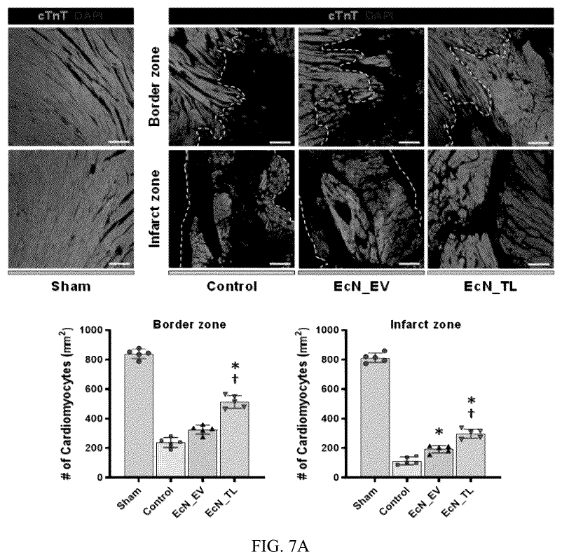

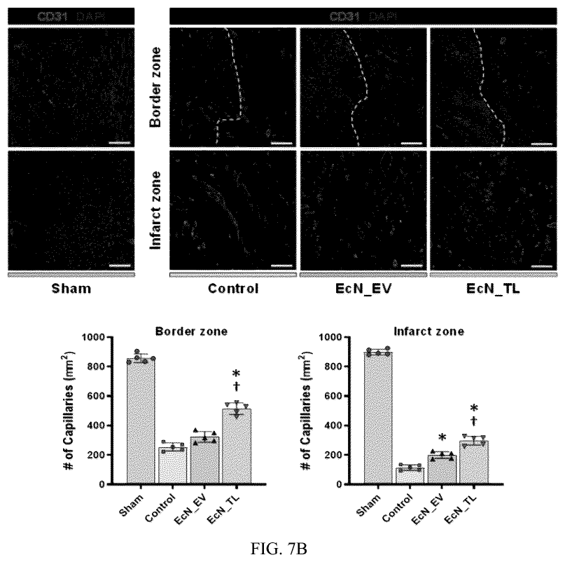

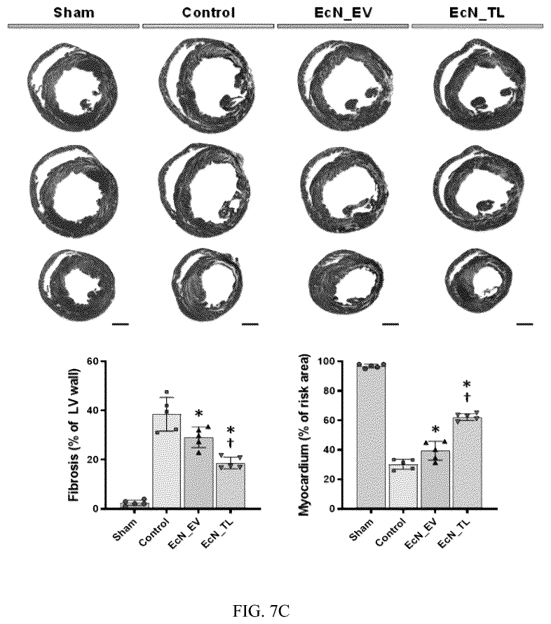

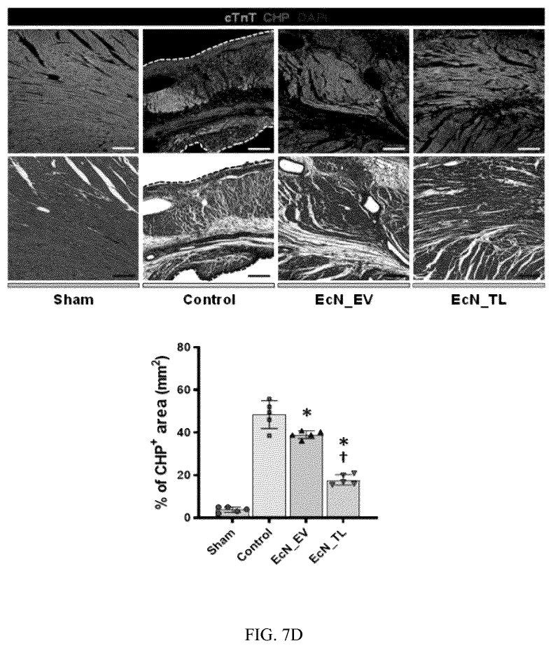

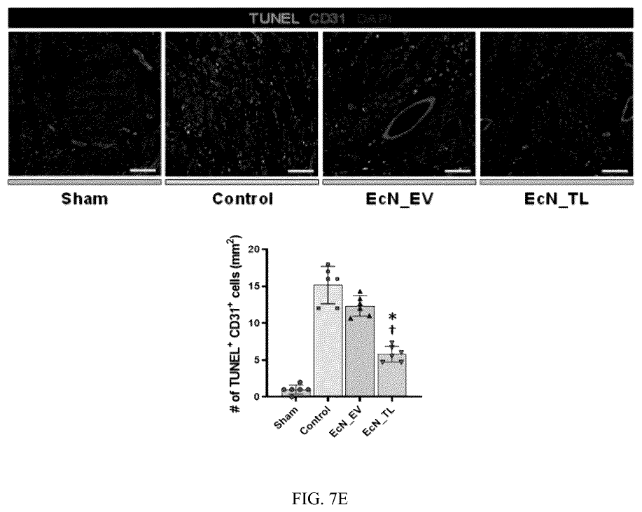

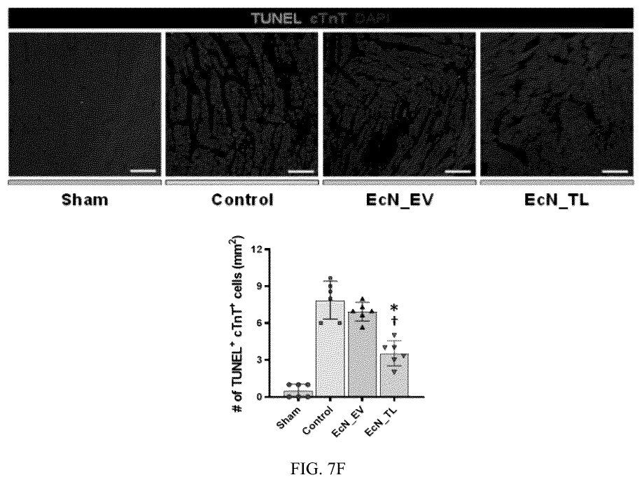

The number of viable cardiomyocytes as determined by troponin T (TnT, a cardiomyocyte specific marker) staining is first assessed. In both the infarct and border zones of the hearts in the EcN_TL-fed rats, there are significantly more viable cardiomyocytes than in other groups (p<0.05) (FIG. 7A). Conversely, the number of TUNEL-positive cells, indicating apoptotic cells in the infarct area, was significantly lower in the EcN_TL group (FIG. 7E and FIG. 7F). Furthermore, the number of small blood vessels, primarily capillaries positive for the endothelial cell marker CD31, in both the border and infarct zones, is greater in the EcN_TL-fed group compared to the other two groups (FIG. 7B). Additionally, Masson's trichrome staining used to quantify cardiac fibrosis shows remarkably lower collagen (blue) and wider viable myocardium (red) in EcN_TL groups compared to both the control and EcN_EV groups (FIG. 7C). Lastly, hybridizing peptide (CHP) staining, which detects degraded collagen, revealed that EcN_TL effectively reduced collagen degradation in the infarct zone compared to the control and EcN_EV groups (p<0.05) (FIG. 7D). These finding are particularly significant because during the acute inflammatory phase post-MI, infiltrating leukocytes phagocytose cell debris and exhibit proteolytic activity. Collagen degradation by these proteolytic enzymes from leukocytes contributes to tissue damage during inflammation. In this regard, these results suggest that EcN_TL may limit the recruitment of inflammatory cells and inflammation-induced myocardial tissue damage.

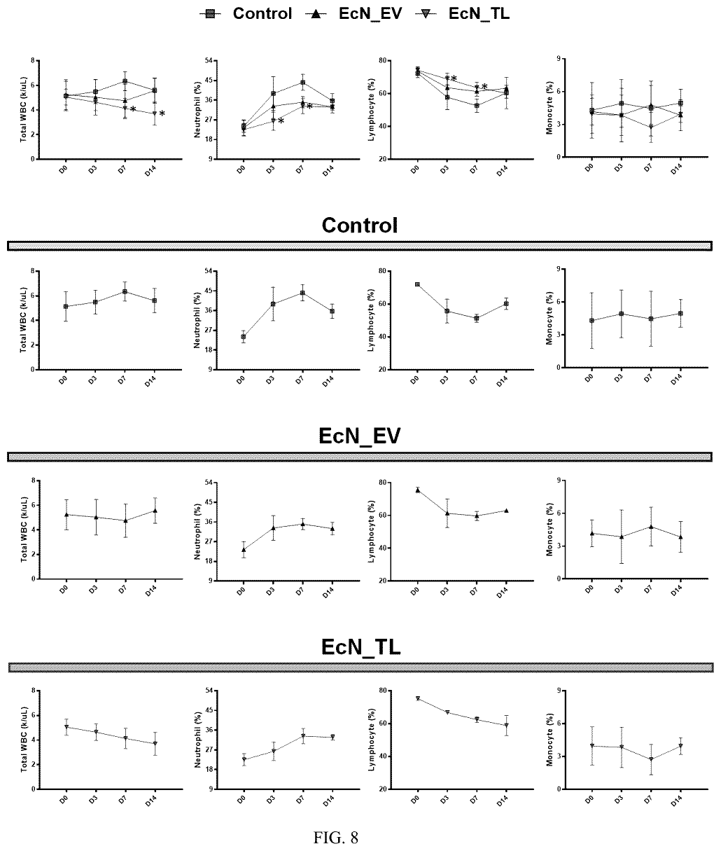

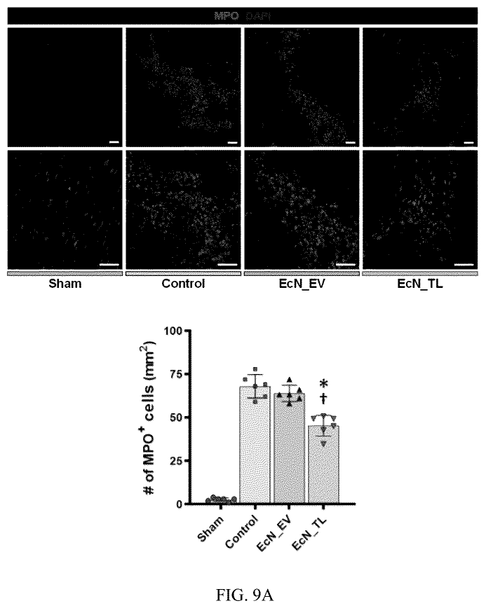

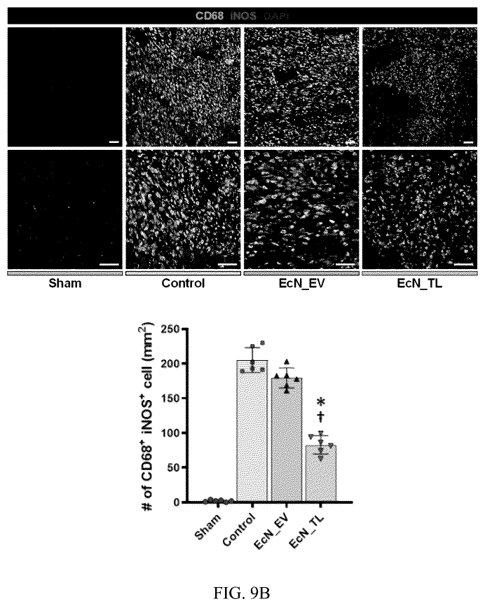

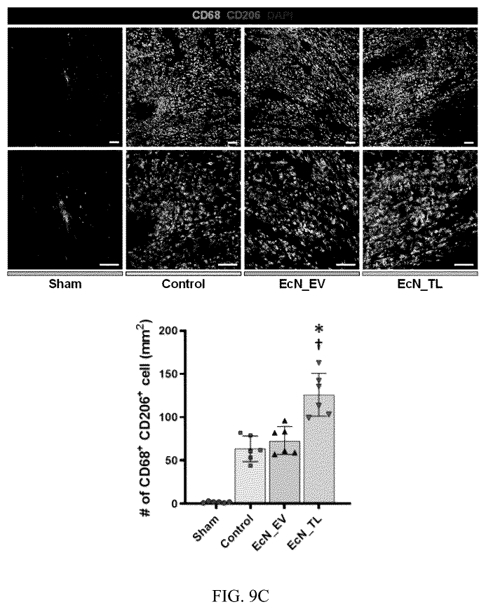

Considering that SCFAs have demonstrated significant anti-inflammatory effects in other studies, together with these results suggesting reduced inflammation-induced myocardial damage, it is hypothesized that EcN_TL protects the heart from I/R injury through its anti-inflammatory effects. To assess systemic inflammation, blood collected from the rats post I/R injury on day 3 and 7 is analyzed. It is found that the percentage of neutrophils, an indicator of systemic inflammation and a potential marker of cardiac injury, is reduced in the EcN_TL-fed group compared to the other groups (FIG. 8). Furthermore, immune staining in heart tissues harvested 4 weeks after I/R injury reveals that the number of neutrophils, detected by myeloperoxidase (MPO) staining, is significantly lower in the EcN_TL-fed group compared to the other groups (FIG. 9). Interestingly, a lower number of iNOS+MI macrophages and a higher number of CD206+M2 macrophages are detected in EcN_TL-treated hearts compared to the other groups (FIG. 9B and FIG. 9C). The recruitment of different phenotypes of macrophages and their subsequent actions play a crucial role in the innate immune response following myocardial injury. During the acute inflammatory phase of MI, the initial wave of infiltrated macrophages promotes clearance of cellular debris and releases pro-inflammatory cytokines, further amplifying inflammation. In the later stages, macrophages transition an anti-inflammatory “M2” phenotype and participate in the inflammation resolution and healing. Taken together, these results evidently indicate that EcN_TL feeding provided extensive benefits, particularly by protecting the heart from I/R injury through its anti-inflammatory effects.

### Example 3. SCFAs Protect Cardiomyocytes Against Inflammation-Induced Injury Via NF-κB Pathway

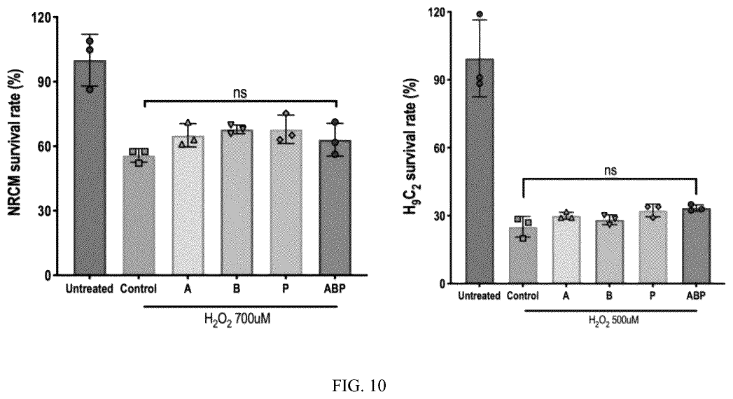

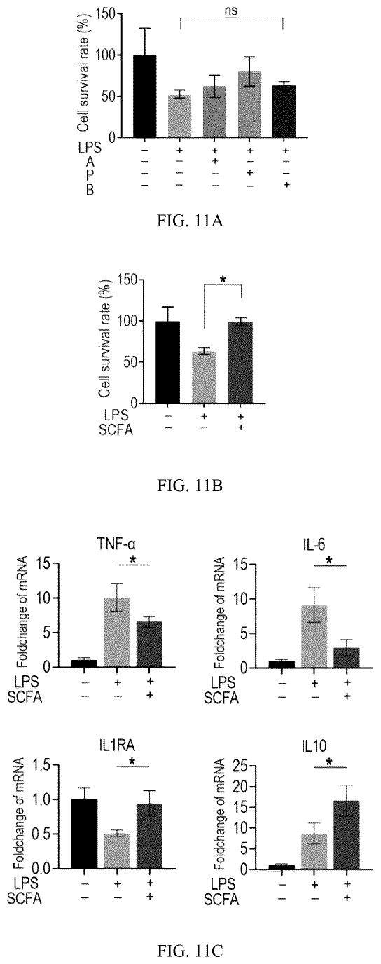

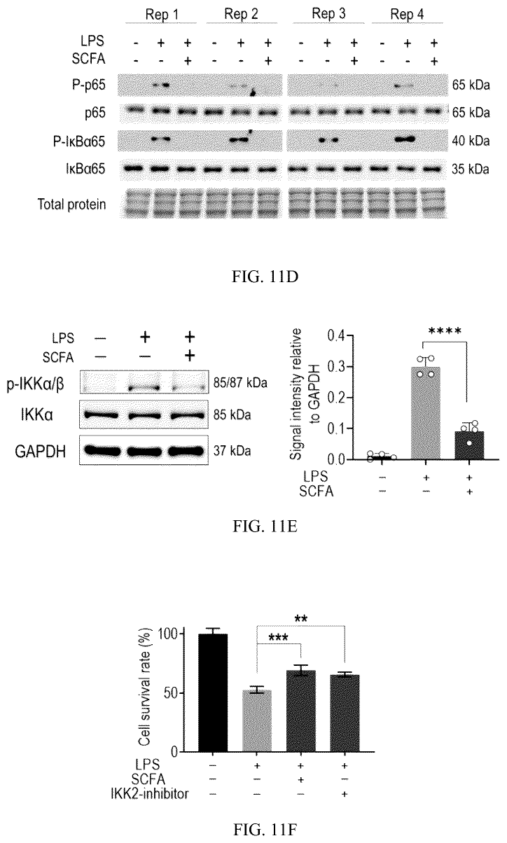

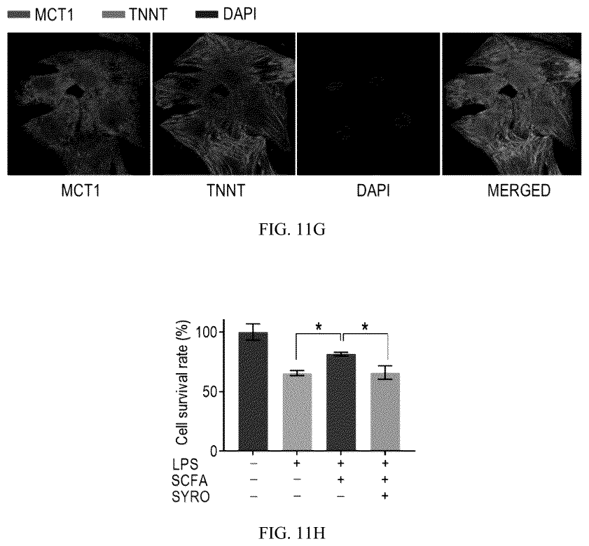

The molecular mechanism underlying SCFA-induced cardio protection is further investigated. While feeding EcN_TL prevented ischemic injury during I/R, it is discovered that SCFAs do not directly protect cardiomyocytes from I/R injury. In vitro experiments using H9C2 myoblasts and primary rat neonatal cardiomyocytes exposed to H₂O₂ to simulate I/R injury show that neither individual SCFAs (acetate, propionate, and butyrate) nor their combination provides protection (FIG. 10). The effect of SCFAs on inflammation-induced injury in cardiomyocytes is further examined. Interestingly, only the combination of SCFAs, but not individually, significantly enhances the survival of cardiomyocytes subjected to lipopolysaccharides (LPS)-induced cell injury (FIG. 11A and FIG. 11B). Moreover, SCFA treatment significantly reduces the mRNA levels of inflammatory cytokines, including TNF-α and IL-6, while promoting the expression of anti-inflammatory cytokines like IL1RA and IL-10 (FIG. 11C).

To investigate the molecular mechanism of SCFAs in cardioprotection, the activation of NF-κB, a central regulator of inflammation that mediates numerous pro-inflammatory gene expressions, including TNF-α, IL-6, and IL-1β, is assessed. By examining the phosphorylation of p65 and IκBa, it is observed that LPS effectively activates NF-κB in cardiomyocytes (FIG. 11D and FIG. 11E). However, consistent with a previous study showing that SCFAs inhibit NF-κB pathway in cells expressing SCFA-sensing receptors such as GPR41 and GPR43, co-treatment of LPS with SCFAs inhibited the phosphorylations of p65, IκBα and IKKα/β without altering the total protein levels. Subsequently, the addition of an IKK2 inhibitor in the cultured cardiomyocytes significantly abolishes the protective effects of SCFA (FIG. 11F).

Furthermore, it is attempted to identify a cardiomyocyte-specific membrane protein that responds to SCFA effects. No known SCFA receptor is found to be expressed in cardiomyocyte. Since SCFAs affect the cells through both membrane receptors and intracellular mechanisms, MCT1, a SCFA transporter, is tested as the potential candidate. Neonatal rat cardiomyocytes are found to express MCT1 on the cell surface as shown by immunohistochemical staining (FIG. 11G). Treatment with an MCT1 inhibitor, Syrospingopine, abolishes the aforementioned protection effect of SCFAs on cardiomyocytes (FIG. 11H). These data strongly suggest that MCTI is a potential SCFA receptor in cardiomyocytes that plays a critical role in SCFA-mediated cardiac protection.

### Example 4. SCFAs Exhibit Anti-Inflammation in Immune Cells

Macrophages and neutrophils are both crucial components of the inflammatory response. In the MI experiments, a decrease in the ratio of MI to M2 macrophages at the infarct site in EcN_TL-fed rats is observed, suggesting that

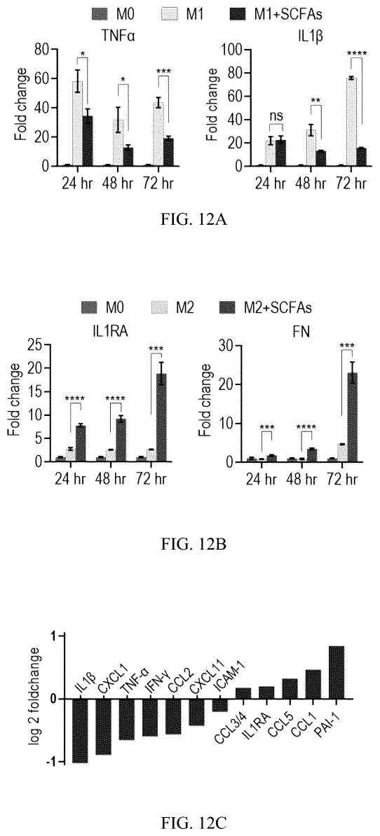

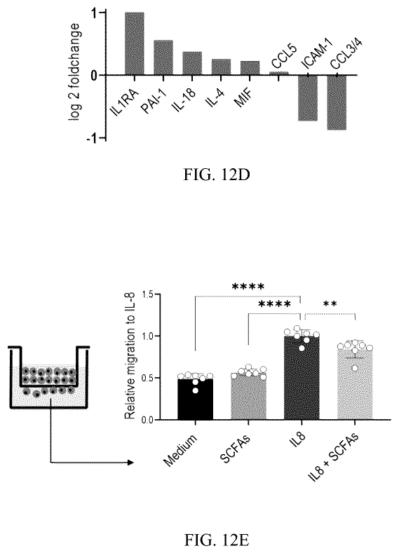

SFCAs may influence macrophage polarization. Therefore, the impact of SCFAs in M1 vs. M2 macrophage polarization is investigated. The human THP-1 differentiated M0 macrophages are differentiated into M1 or M2 phenotype in the presence of a mixture of SCFAs, including acetate, propionate, and butyrate. During M1 differentiation, SCFAs treatment suppresses the expression of inflammatory cytokines TNF-α and IL-1β (FIG. 12A), indicating a dampening effect on the inflammatory phenotype of MI macrophages. In contrast, SCFA treatment enhances the expression of IL1Rα, an IL-1 antagonist known to reduces inflammation. The expression of FN (fibronectin-1), important for the wound healing function of M2 macrophages, is also elevated upon SCFA treatment (p<0.001) (FIG. 12B). To further investigate the broader effect of SCFAs on cytokine production, a cytokine array is utilized to analyze the protein levels of a panel of cytokines in the supernatant of M1- or M2-differentiated macrophages. Briefly, cell-free supernatants from macrophages are collected after 72 hours of incubation and assessed for cytokine expression using a semiquantitative immunosorbent approach with Proteome Profiler Human Cytokine Array Kit/ARY005 (R&D Systems).

The results show that SCFAs downregulate the expression of pro-inflammatory cytokines (IL1β, CXCL-1, TNF-α, IFN-γ, and CCL-2) in M1 macrophages, while stimulating the production of anti-inflammatory molecules (IL1RA, PAI-1, and IL-4) in M2 macrophages (FIG. 12C and FIG. 12D). These findings indicate that SCFAs modulate macrophage polarization and function toward an anti-inflammatory phenotype.

Next, the effect of SCFAs on neutrophil migration induced by IL-8, a chemokine expressed by cardiomyocytes after MI, is examined. In a modified Boyden Chamber transwell system, neutrophils in the upper compartment migrate toward the chemoattractant IL-8 in the lower compartment, while medium or SCFAs alone induces minimal neutrophil chemotaxis. The presence of SCFAs at serum concentration inhibits IL-8-induced neutrophil migration, suggesting a direct effect of SCFAs on neutrophil chemotaxis (FIG. 12E).

During inflammation, neutrophils bind to the adherent molecules highly expressed in endothelial and roll along the vessel wall, following chemoattractants released from the injured myocardium. It is found that SCFAs protect human endothelial cells (HUVEC) from inflammatory injury, suggesting that SCFAs dampen the inflammatory response in endothelial cells expressing the receptor GRP41.

The results demonstrate that the combination of acetate, propionate, and butyrate at physiological plasma ratio suppresses the inflammatory response in endothelial cells, inhibits IL-8-dependent neutrophil chemotaxis, reduces the pro-inflammatory M1 phenotype, and promotes the anti-inflammatory wound healing M2 macrophages.

In summary, the present invention demonstrates that the engineered probiotic, EcN_TL, continuously and consistently secretes a physiological level of SCFAs into the bloodstream. EcN_TL is created by introducing two biosynthetic pathways, propionate and butyrate, into the EcN. When it is fed to rats for one week, EcN_TL effectively protects the animals from subsequent myocardial I/R injury, leading to a significant improvement in left ventricular heart function and ameliorating adverse cardiac remodeling and fibrosis. Moreover, the SCFAs protect the cardiomyocytes from cell death induced by inflammation, although they do not have a significant effect on ischemic stress. Additionally, SCFAs exhibit anti-inflammatory effects on immune cells.

Consistent with previous studies that demonstrate significant anti-inflammatory effects of SCFAs on macrophages, the present invention shows that oral treatment of EcN_TL exerts cardioprotective effects through both immune cell-dependent and -independent mechanisms, primarily by reducing inflammation. SCFAs directly suppress pro-inflammatory cytokine production in cardiomyocytes by inhibiting the activation of NF-κB, a key transcription factor for the production of various pro-inflammatory cytokines that exacerbate adverse cardiac remodeling and contribute to heart failure. Indeed, NF-κB inhibition has been proposed as a promising target for treating MI. In addition to reducing pro-inflammatory cytokines in MI hearts, EcN_TL also upregulates anti-inflammatory cytokines such as IL1RA and IL-10. Cardiomyocytes do not express any known SCFA receptor. It is discovered that the effect of SCFAs is dependent on MCTI expression, suggesting a potential role of MCTI as a novel SCFA receptor.

SCFAs have been found to exert a direct influence on immune cells, particularly in the context of cardiovascular health. Previous studies have demonstrated that propionate, one of the key SCFAs, targets regulatory T cells, modulating inflammatory responses, balancing T helper cell homeostasis, and reducing immune cell infiltration, all of which contribute to mitigating hypertensive cardiac damage and atherosclerosis. The present invention, however, focuses on the effects of SCFAs on innate immune cells, which are crucial in initiating and modulating the inflammatory response.

In particular, SCFAs have been shown to promote the polarization of macrophages towards the M2 phenotype, both in vivo in myocardial infarction (MI) hearts and in vitro models. When animals are fed with EcN_TL, there is an observed increase in M2 macrophages and a corresponding decrease in M1 macrophages within the infarct zone of MI hearts. This polarization towards the M2 phenotype is associated with an increased expression of anti-inflammatory molecules and a decreased production of pro-inflammatory cytokines in macrophages. Additionally, SCFA treatment reduces neutrophil infiltration into the MI heart in vivo and inhibits neutrophil migration in vitro, further underscoring the role of SCFAs in modulating the inflammatory response.

These findings strongly support the role of SCFAs in regulating macrophage polarization towards a wound-healing phenotype, minimizing neutrophil-induced inflammatory injury, and creating a microenvironment that fosters tissue repair in the MI heart. Moreover, prior research has indicated that butyrate, another SCFA, modulates the expression of genes related to inflammation and apoptosis, helping to maintain balance in the gut-heart axis. The present invention builds upon this foundation, expanding the understanding of SCFAs' immune effects and elucidating both immune cell-dependent and -independent mechanisms through which SCFAs exert cardioprotective effects.

While diets supplemented with SCFAs have been shown to provide benefits in heart diseases prevention and protection, maintaining effective SCFA levels in the bloodstream remains a critical challenge. The concept of using probiotics to release SCFAs is introduced a decade ago, but an effective probiotic treatment for MI has not yet been developed. A recent study on the relationship between the microbiome and cardiac repair highlighted the role of SCFAs in post-MI recovery. The engineered probiotic, EcN_TL, of the present invention possesses the ability to constantly release SCFAs and maintain their concentration in the bloodstream during acute MI, thereby reducing of cardiomyocyte death and fibrosis, improving contractibility of left ventricular and enhancing cardiac performance.

In conclusion, the present invention provides a strong basis for utilizing synthetic live bacterial therapeutics to prevent heart diseases and laid the foundation of engineered probiotics as bio-therapeutic agents that target the gut-heart axis. Beyond protecting against coronary heart diseases, the engineered probiotic EcN_TL, which provides constant delivery of SCFAs, can be applied to other health conditions. As a tool, it can be used to probe the role of SCFAs in the bidirectional communication of gut-related axes.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

1. A genetically engineered Escherichia coli Nissle 1917 (EcN) probiotic strain, comprising a modified genome, wherein the modified genome is modified to comprise:
an atoB gene, derived from E. coli K-12, encoding acetyl-CoA acetyltransferase and having a sequence of SEQ ID NO: 01;
a crt-bcd-etfA-etfB-BHBD gene cluster, derived from E. C. butyricum, encoding enoyl-CoA hydratase, butyryl-CoA dehydrogenase, alpha and beta subunits of electron transfer flavoprotein, and 3-hydroxybutyryl-CoA dehydrogenase and having a sequence of SEQ ID NO: 02; and
a ptb-buk gene, derived from C. acetobutyricum, encoding phosphotransbutyrylase and butyrate kinase and having a sequence of SEQ ID NO: 03;
wherein ldhA, frdABCD, adhE, ackA and pta genes are deleted from the modified genome.

2. The genetically engineered EcN probiotic strain of claim 1, wherein the genetically engineered EcN probiotic strain constantly secretes short-chain fatty acids (SCFAs) and elevates the blood concentration of the SCFAs in the subject.

3. The genetically engineered EcN probiotic strain of claim 2, wherein the SCFAs comprise propionate and butyrate.

4. The genetically engineered EcN probiotic strain of claim 1, wherein the deletion of the ldhA, frdABCD, adhE, ackA, and pta genes minimizes the production of competing metabolic byproducts, thereby increasing the efficiency of butyrate synthesis.

5. The genetically engineered EcN probiotic strain of claim 4, wherein the competing metabolic byproducts comprise lactate, ethanol, succinate, and acetate.

6. The genetically engineered EcN probiotic strain of claim 1, wherein the ldhA gene has a sequence of SEQ ID NO: 04, the frdABCD gene has a sequence of SEQ ID NO: 05, the adhE gene has a sequence of SEQ ID NO: 06, the ackA gene has a sequence of SEQ ID NO: 07 and the pta gene has a sequence of SEQ ID NO: 08.

7. A composition for treating a coronary heart disease in a subject in need thereof, comprising a genetically engineered EcN probiotic strain and a pharmaceutically acceptable carrier;
wherein the genetically engineered EcN probiotic strain has a modified genome, the modified genome is modified to comprise:
an atoB gene, derived from E. Coli K-12, encoding acetyl-CoA acetyltransferase and having a sequence of SEQ ID NO: 01;
a crt-bcd-etfA-etfB-BHBD gene cluster, derived from E. C. butyricum, encoding enoyl-CoA hydratase, butyryl-CoA dehydrogenase, alpha and beta subunits of electron transfer flavoprotein, and 3-hydroxybutyryl-CoA dehydrogenase and having a sequence of SEQ ID NO: 02; and
a ptb-buk gene, derived from C. acetobutyricum, encoding phosphotransbutyrylase and butyrate kinase and having a sequence of SEQ ID NO: 03;
wherein ldhA, frdABCD, adhE, ackA and pta genes are deleted from the modified genome;
wherein the genetically engineered EcN probiotic strain constantly secretes propionate and butyrate and elevates the blood concentration thereof in the subject.

8. The composition of claim 7, wherein the pharmaceutically acceptable carrier is selected from a diluent, a cryoprotectant, an encapsulation agent, a stabilizer, a buffer, an antioxidant, a preservative, a disintegrant, a binding agent, a coating agent, a liquid carrier, or a food-based carrier.

9. The composition of claim 7, further comprising a dietary fiber source that enhances the production of propionate and butyrate by the genetically engineered EcN probiotic strain.

10. The composition of claim 7, wherein the genetically engineered EcN probiotic strain is encapsulated in a biocompatible polymer to improve its viability during storage and delivery.

11. The composition of claim 7, wherein the pharmaceutically acceptable carrier is formulated for oral administration in the form of a capsule, tablet, powder, or liquid suspension.

12. The composition of claim 7, wherein the ldhA gene has a sequence of SEQ ID NO: 04, the frdABCD gene has a sequence of SEQ ID NO: 05, the adhE gene has a sequence of SEQ ID NO: 06, the ackA gene has a sequence of SEQ ID NO: 07 and the pta gene has a sequence of SEQ ID NO: 08.

13. A method of treating a coronary heart disease in a subject in need thereof, comprising orally administering a therapeutically effective amount of the pharmaceutical composition of claim 6 to the subject.

14. The method of claim 13, wherein the coronary heart disease is selected from myocardial infarction, atherosclerosis, angina pectoris, coronary artery disease, or ischemic heart disease.
